(19) United States
(12) Patent Application Publication
(10) Pub. No.: US 2009/0137402 A1
(43) Pub. Date: May 28, 2009

(54) Ditag genome scanning technology

(76) Inventors: San Ming Wang, Northbrook, IL (US); Jun Chen, Nanjing City (CN); Yeong Cheol Kim, Wilmette, IL (US)

Correspondence Address: LEYDIG VOIT & MAYER, LTD, 700 THIRTEENTH ST. NW, SUITE 300, WASHINGTON, DC, 20005-3960, US

(21) Appl. No.: 11/907,404

(22) Filed: Oct. 11, 2007

Related U.S. Application Data
(60) Provisional application No. 60/850,648, filed on Oct. 11, 2006.

(51) Int. Cl. C40B 20/00; C12Q 1/68; G06F 17/30

(52) U.S. Cl. 506/2; 435/6; 707/3; 707/E17.108

(57) ABSTRACT

The present invention provides for a method for analyzing large genomes using a process by where the genomic DNA is digested by a small base pair restriction enzyme. The fragments are then cloned and a unique ta-vector-tag is created. The tag-vector-tag fragments are purified and re-ligated to create a “ditag” library, which are then sequenced. In the final step, the sequenced ditags can be mapped back to the genome using software containing mapping algorithms and a unique ditag reference database to provide a method for scanning large portions of the genome in a reduced amount of time and cost.

This invention claims priority to U.S. Provisional Application Ser. No. 60/850,648, filed Oct. 11, 2006, and is hereby incorporated by reference in its entirety as if set forth herein.

### BACKGROUND OF THE INVENTION

1. Field of Invention

This invention relates to the field of gene sequencing. More specifically, this invention relates to high throughput genome sequencing and mapping, and its use to identify potential genome variations based on mapping information.

2. Description of Prior Art

Studying human genome structure provides clues for understanding fundamentals in biology, and for identification of genetic abnormalities related to human diseases. While the accomplishment of the human genome project has opened the path (1-2), only a few individual genomes have been analyzed thus far. Increasing evidence suggests wide variation among different individual genomes (3-8). Therefore, a large number of individual human genomes need to be analyzed in order to fully understand the genome (9).

Because of the large size of most mammalian and human genomes and the limited power of current technologies, analyzing multiple genomes remains challenging. Potential tools for the study include the array-based approach such as Comparative Genomic Hybridization (array-CGH) and genome tilling-array (10-13) and whole genome sequencing-based approaches. Taking advantage of the completed human genome sequences as the reference for probe designing, the array-based approach provides high-throughput capacity, robust, industrial standard and sensitivity to detect copy number changes in the genome.

The array-based approach has limited power for identifying structural variations such as insertion, inversion and translocation, nor does it detect repetitive regions and unknown DNA. The sequencing-based approach provides direct sequence information for the detected DNA. As an open system, it detects both known and unknown DNA without the need of a priori knowledge of the genome contents. This feature is critical for studying normal genome variations and characterizing disease genomes. However, the high cost, at over $10 million per human genome, of using the current Sanger sequencing system prevents its routine use in sequencing multiple genomes. While attempts are underway to substantially decrease the sequencing cost and increase the throughput-capacity (14-16), fully sequencing multiple human genomes presently remains impractical.

The recently developed 454 sequencing system (454 Life Sciences, Inc. Branford, Conn. 06405) has significantly increased the throughput-capacity and decreased the cost of DNA sequencing collection (16). The system analyzes over 20 Mb per run, which is about 1/150^{th} of the human genome size, at the direct cost of less than $10,000 per sequencing run. However, the current 454 system can only handle microbial genomes at Mb sizes, not large genomes like the human genome.

However, there still exists a need for a method of simplifying large genomic DNA in such a way that provides a method that is capable of being used in human diagnostic applications.

### SUMMARY OF THE INVENTION

In accordance with the present invention, it has been discovered by Applicants, that they could take advantage of the 454 sequencing system for large genome study. Applicants' approach was to simplify the large-size genome into a smaller one in order to meet the capacity of the 454 sequencing system. This was achieved by collecting short tags across the whole genome. Applicants have named their approach the Ditag Genome Scanning (DGS) system.

The main components of DGS comprise: 1) collecting two short tags from both ends of DNA fragments to form a ditag; 2) using the 454 sequencing system for maximal collection of ditags at the genome scale; 3) identifying the DNA fragments in the human genome sequences that originated the ditags and identify the DNA fragments that are different from those in the reference human genome; 4) confirming the mapping results by using the ditag sequences directly as the sense and antisense primers in a PCR expansion to detect the original DNA fragments; 5) performing computational and experimental analysis of DGS results. The present DGS invention has many unique features for analyzing genome structure.

The DGS process can be described in the following steps: a genomic DNA sample is digested by a restriction enzyme. The digested genomic DNA fragments are cloned into vectors to generate a genomic DNA library. The library is then digested by MmeI, which retains two short tags on each site in the same vector. The tag-vector-tag fragments are gel-purified and re-ligated to form a ditag library. Ditags are released from the vectors and concatemerized at random orientations. The concatemerized ditags are then sequenced by using the 454 DNA sequencing system. The resulting ditag sequences are mapped in either sense or antisense orientation to the ditag reference database constructed from the human genome sequences. The mapping result is confirmed by PCR using each single tag in the ditag sequences as the sense and antisense primers.

The invention, together with other objects and advantages, which will become subsequently apparent, reside in the details of the technology as more fully hereinafter described and claimed, reference being had to the accompanying drawings forming a part hereof, wherein like numerals refer to like parts throughout.

### BRIEF DESCRIPTION OF THE FIGURES

FIG. 1A is an illustration of the overall DGS system process.

FIG. 2 shows the potential variations detectable by DGS ditags. (A) A normal ditag represents a normal DNA fragment in the genome. (B) Deletion. One tag maps in the expected site but the second tag maps a distal tag far from the expected restriction site. (C) Inversion. One of the tags maps to a neighboring tag but in reverse orientation. (D) Insertion. One tag maps but the other tag maps to a tag interior to the expected location. (E). Translocation. Each single tag in a ditag maps to different chromosomes.

FIG. 3 shows the size distribution of DNA fragments detected by the experimental ditags. The experimental ditags from Kasumi-1 cell were mapped to the reference ditag database. Those DNA fragments contributed the mapped reference ditags were assigned as the original fragments for the experimental ditags. The size distribution of those DNA fragments was compared with that of the DNA fragments in the reference human genome sequences.

FIG. 4 is a karyotype of Kasumi-1 cells. The picture shows that the genome of kasumi-1 cell is significantly different from the normal genome. 48,X,−Y,+3, add(7)(p11.2),t(8;21)(q22;q22),I(9)(q10),der(12)t(2;12)(q31;p13),13, add(15)(p11.2),−16, add(19)(p13.3),+mar2,+mar3×2[20].

FIG. 5a shows an example of experimental confirmation of ditag mapping.

FIG. 5b shows another example of experimental confirmation of ditag mapping.

FIG. 5c shows a further example of experimental confirmation of ditag mapping.

FIG. 6 presents a table analyzing the total bases from 6-mer restriction fragments of the human genome.

FIG. 7 presents a table showing the distribution and specificity of SacI DGS ditags in the human genome.

FIG. 8 presents a table describing the length and genome coverage of DNA fragments by 6-mer restriction enzymes.

FIG. 9 presents a table displaying and analysis of the collection of ditags by using 454 sequencing system.

FIG. 10 presents a table showing how ditags were mapped to reference ditags of Y chromosome.

FIG. 11 presents a table showing a summary of PCR confirmations for 145 selected ditags.

FIG. 12 presents a table showing the classification for the trouble mapped ditags.

FIGS. 13a-c presents a table showing the experimental confirmation of ditag mapping results.

FIG. 14 is a schematic illustration of the DGS process of Example 4.

FIG. 15 shows the size distribution of DNA fragments of the virtual SacI DNA fragments from HG18, and the virtual DNA fragments in HG18 mapped by GM1510 SacI ditags.

FIG. 16 is a schematic example of the variations detected by a ditag. This variation was identified in fosmid sequence (TI number 146956937). Its 5′ part contains a 24-base insertion including the restriction site that is not present in HG18.

FIG. 17 is a diagrammatic representation of genome variations in four individual genomes detected by ditags. The ditags not mapped to the HG18 were mapped against the reference ditags from the four human genome sequences of the fosmid sequences, the Celera genome sequences, the Venter genome sequences and the Watson genome sequences. The figure shows the distribution of the mapped ditags among the four individual genomes.

FIG. 18 is an example of the insertion confirmed by ditags. Variation AC153461 contains an 8002 bp insertion. Six ditags detected this insertion, of which 4 were within the insertion, and 2 crossed the junctions between the normal sequences and the insertion in the SacI restriction site.

FIG. 19 shows the results of ditag-detected genome variations in multiple individual genomes. Variations in detected by 5 SacI ditags were tested in a panel of 10 DNA samples (Coriell). GM: GM15510 DNA was used as the positive control. The results show that two variations are present in all ten individual genomes and three variations only exist in 4 individual genomes.

### DETAILED DESCRIPTION AND PREFERRED EMBODIMENTS

In describing embodiments of the invention, specific terminology will be resorted to for the sake of clarity. However, the invention is not intended to be limited to the specific terms so selected, and it is to be understood that each specific term includes all technical equivalents which operate in a similar manner to accomplish a similar purpose.

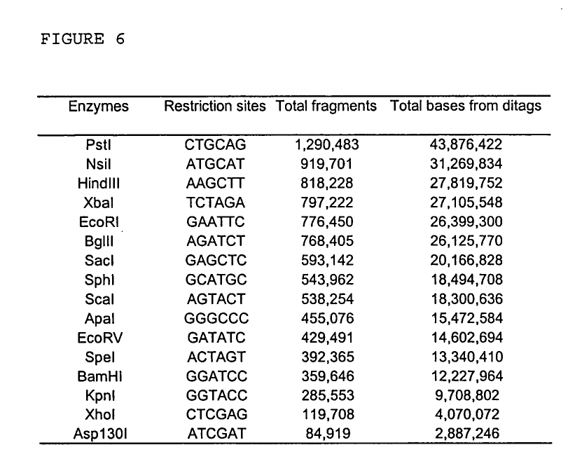

Using the human genome sequences as the reference, Applicants analyzed the relationship between the ditag and the human genome. The number of restriction fragments generated by different restriction enzymes varies. Consequently, the total number of bases from the ditags from each type of restriction fragments differs. The total number of bases from ditags of the fragments generated by the 6-mer restriction enzymes is between about from 1 Mb to 100 Mb, and within our data between about 2.9 Mb to 43.9 Mb (FIG. 6). This size range is the range to be covered by the 454 DNA sequencing system. Taking SacI fragments as the example, there are 593,142 ditags from the same number of SacI DNA fragments in the human genome. Those ditags contain 20,166,828 bases that is a 149-fold size reduction of the human genome sequences (3 Gb of the human genome: 20 Mb of ditags=149:1). A single run of 454 sequencing collection can provide 1× coverage of the total bases of ditags from these fragments if each ditag is sequenced once.

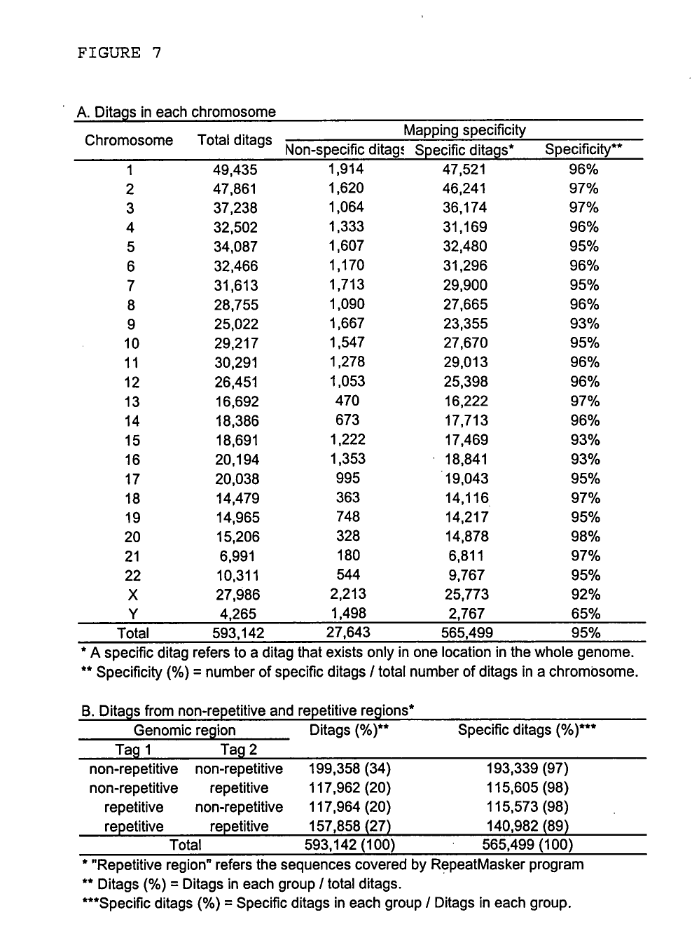

A ditag is comprised of the two ends of a single DNA fragment. Upon releasing from the two ends of the DNA fragment, the two tags form a ditag, which stays together as a single unit during all downstream experimental steps. Until the work by the present inventors, it was unclear to those of ordinary skill in the sequencing art, that when mapping the ditag sequences to the genome, that these two tags actually do map specifically to the two ends of the original DNA fragment. The mapping of the virtual ditags to the virtual restriction fragments of the human genome sequences shows that this is indeed the case. For example, there are 573,941 unique ditags from 593,142 SacI fragments of the human genome sequences. Of these unique ditags, 565,499 (95%) map uniquely to the original DNA fragments. The high specificity is rather consistent for each chromosome except Chromosome Y (FIG. 7). The high specificity of the DGS ditag is also reflected for the repetitive sequences. Half of the human genome is composed of repetitive DNA. Analysis of the 593,142 SacI ditags extracted from the human genome sequence shows that 40% of the ditags are from the boundary between non-repetitive and repetitive DNA (one tag in the non-repetitive region and the other tag in the repetitive region), and 27% are from the pure repetitive DNA. Mapping results disclosed herein show that 98% of ditags from the boundary, and 89% of the ditags from the pure repetitive DNA are specific (FIG. 7b). The high specificity of DGS ditags representing the repetitive DNA fragments provides a powerful means for analyzing the structure of the repetitive regions in the genome.

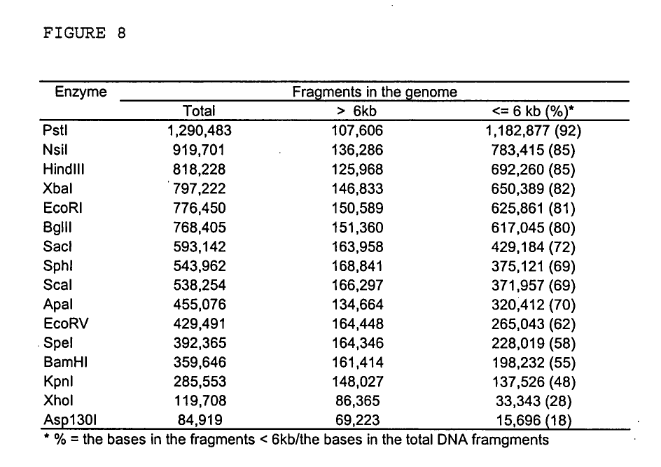

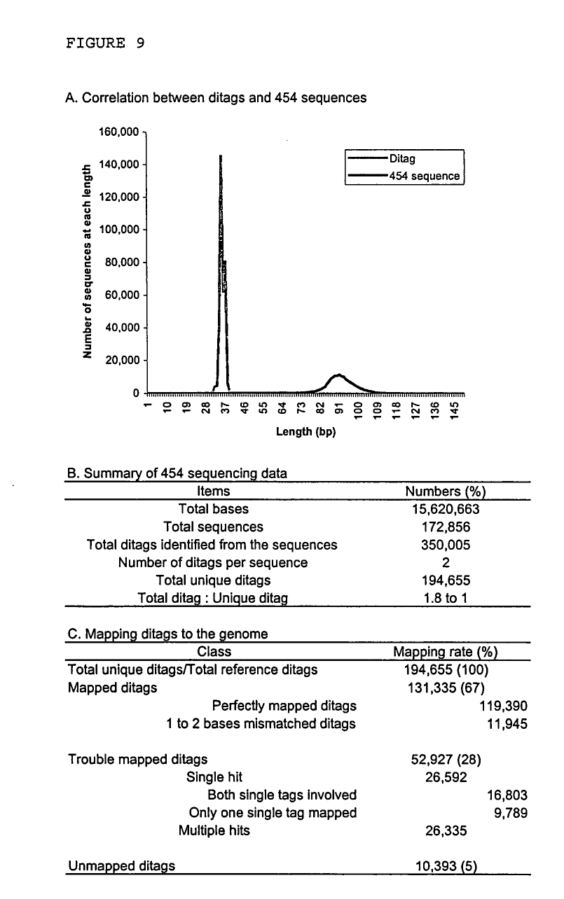

Of the fragments generated by the 6-mer restriction enzymes, the number of fragments longer than 6 kb is rather constant, with less than 2-fold variations, but the fragments shorter than 6 kb vary 75-folds (FIG. 8). Thus, by selecting different restriction enzymes, the inventors are able to target smaller fragments with higher frequency. For example, of the 593,142 SacI fragments, 72% (429,184) are shorter than 6 kb. The large amount of ditags from shorter DNA fragments provides high resolution for scanning the genome.

### Ditags can be Used to Identify Different Types of DNA Structural Variations

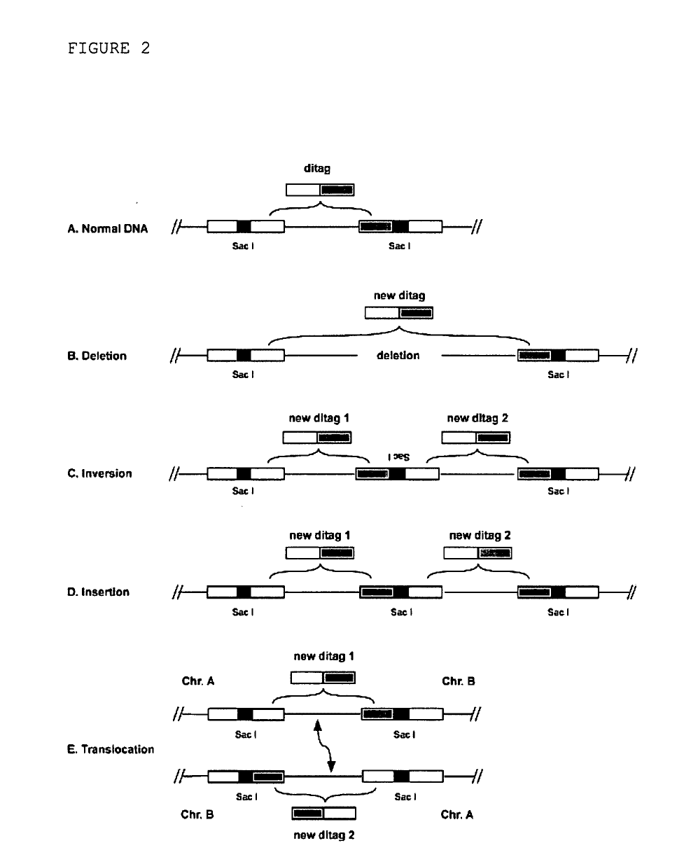

The two tags provide high specificity to represent their original DNA fragment. A DNA fragment that is different from that of the reference human genome sequences is readily distinguishable since its corresponding ditag has no match in the reference ditag database. Those ditags can be further classified into the subtypes, including deletion, inversion, insertion, and translocation (FIG. 2). For the ditag that maps nowhere in the reference ditag database, it may represent unknown genomic DNA fragment that is not included in the reference human genome sequences.

### Experimental Analysis of DGS Ditags

Using the DGS protocol, we constructed a DGS ditag library by using SacI restriction fragments of the leukemia Kasumi-1 cell (17), collected the ditag sequences by using the 454 sequencing system, and analyzed the ditag data by referring to a ditag reference database based on the human genome sequences. Karyotyping analysis of Kasumi-1 cell confirmed its complicated genome structure (FIG. 4).

### Collecting and Mapping Ditag Sequences

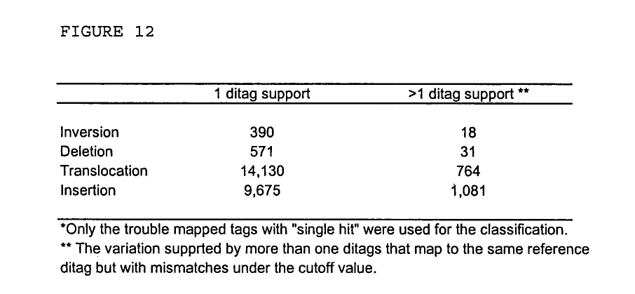

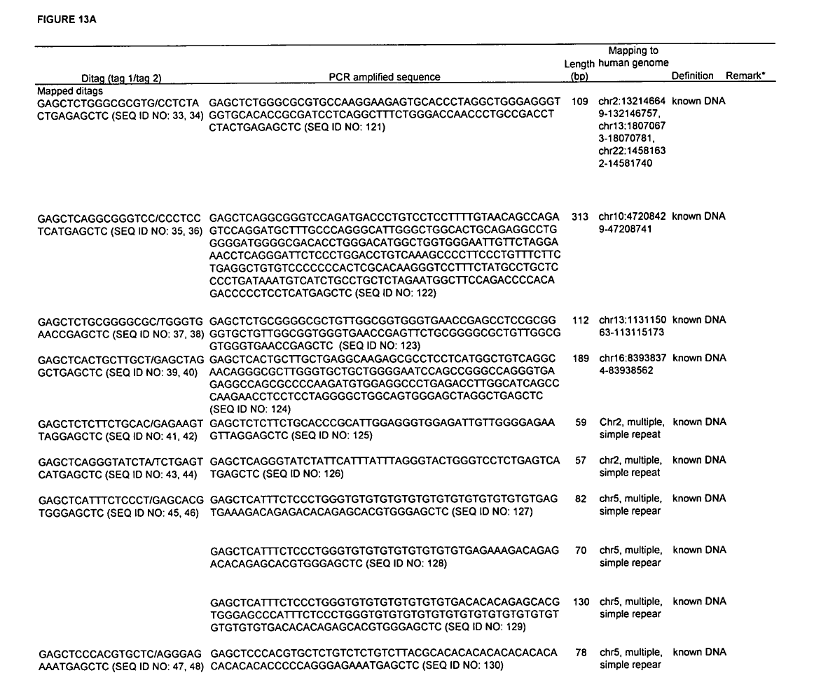

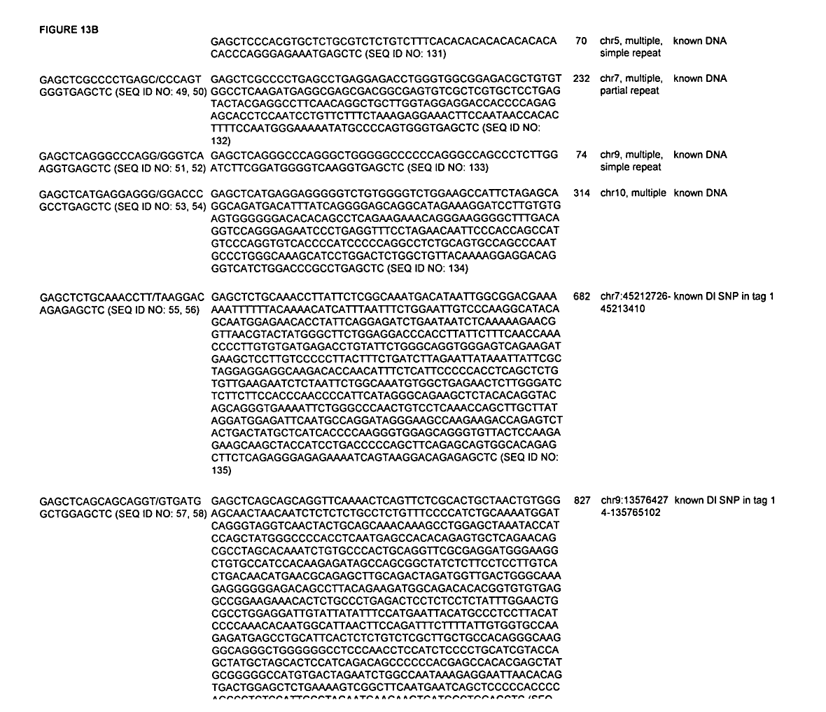

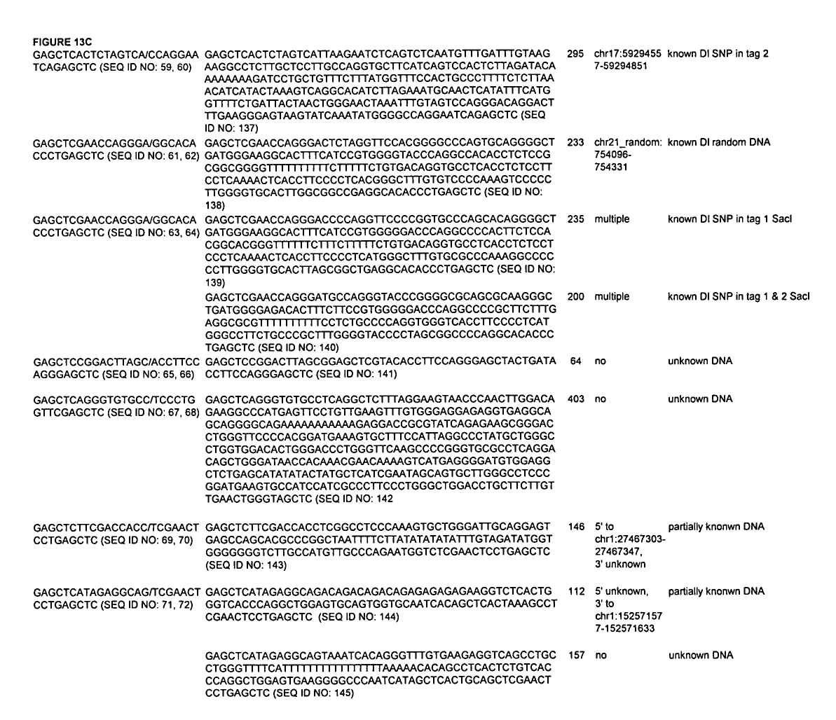

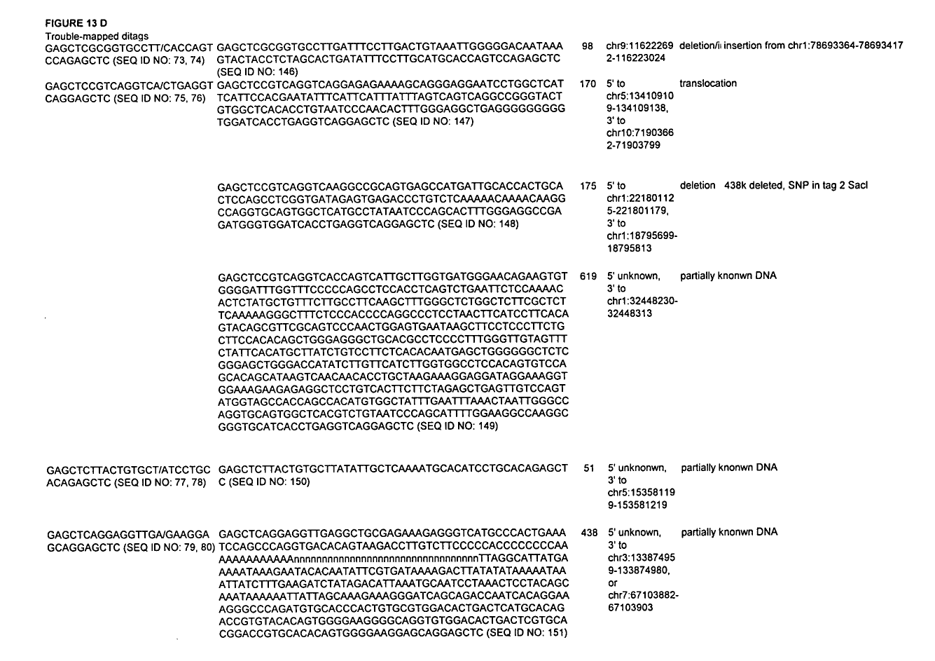

Ditag sequences were collected by a single run of the 454 sequencing system. The length of 454 sequences is distributed between about 40 to 150 bp, with the median length of 90 bp. The length of extracted ditags from the sequences is dominantly distributed between about 28 to 40 bp (FIG. 9). A total of about 350,005 ditag copies were collected from the set of 454 sequences, with the average of two ditags collected per sequence. This equals to 0.59× coverage of the genome ditags, or 0.82× coverage of the fragments shorter than 6 kb. From the collected 350,005 ditag copies, 194,655 unique ditags are identified (FIG. 9). Mapping the experimental ditags to the ditag reference database shows that 67% of the ditags map to the reference ditags, 28% show various types of trouble mapping, and 5% have no mapping in the genome (FIGS. 9, 12).

### Ditag Detects Shorter DNA Fragments

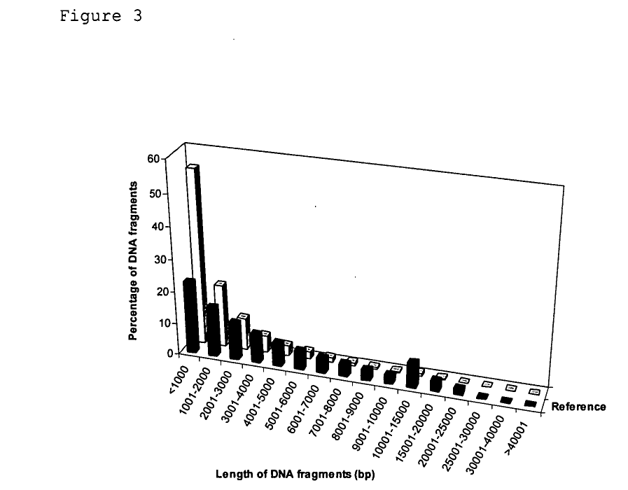

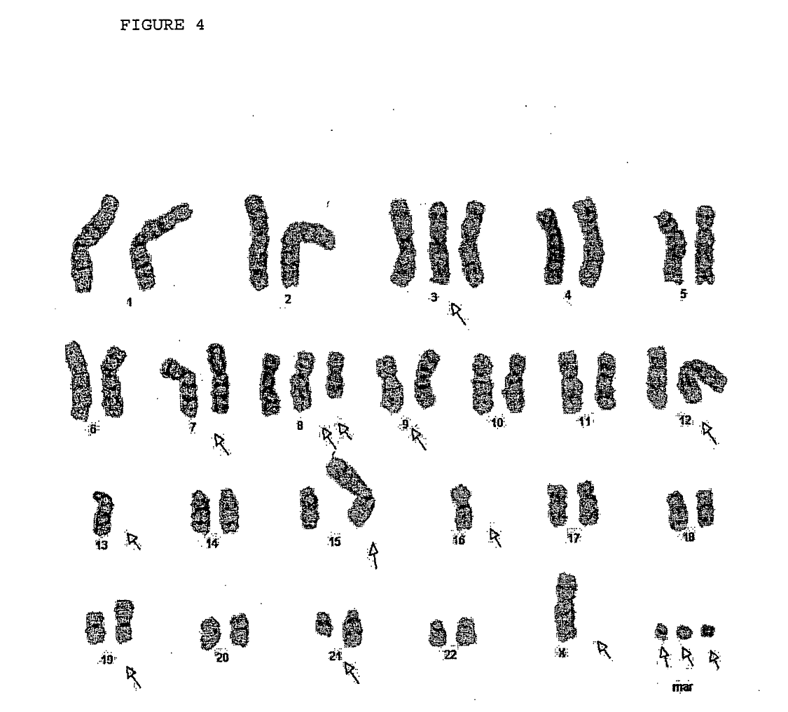

In order to provide high-resolution for genome scanning, DGS uses plasmid as the vector in order to clone DNA fragments of small sizes. The length distribution of the reference fragments mapped by the inventors using experimental ditags shows that 53% of the detected fragments are shorter than 1 kb and 96% of the fragments are shorter than 6 kb, compared to 23% and 77% in the human genome sequences, respectively (FIG. 3). The average length of fragments detected by DGS ditags is 1,665 bps. This information confirms that DGS indeed provides high resolution for genome scanning.

### Ditags Detect DNA Fragments Different from those of the Reference Human Genome

The ditags that map with reference ditags represent identical DNA fragments between the tested sample and the reference human genome. Those ditags that do not have the counterpart reference ditags in the reference database, represent the DNA fragments that are potentially different from those of the standard human genome sequences. Considering that such events could also be related to the mismatches between the experimental ditag with the reference ditag, due to SNP differences between the testing genome and the reference human genome, or sequencing error in the ditag sequences, the inventors set the p value of 1.0e-5 as the cut-off to determine if an experimental ditag has a counterpart in the reference ditag database. Ditags with a p-score less than the cutoff are considered to have no counterpart in the reference database. Such ditags represent potential structural variation including deletion, insertion, inversion, and translocation, and SNPs/sequencing errors (FIG. 9).

### Ditag Detects DNA Fragments not Included in the Human Genome Sequences

The inventors found that a total of 10,393 ditags have no map in the reference ditag database. The Kasumi-1 cell line was established by natural growth as opposed to the commonly used EBV transformation (17). Therefore, these unmapped ditags are not from the EBV genome as confirmed by negative mapping of these ditags to EBV genome. Mapping to the E. coli genome identified only four ditags, ruling out the possibility of experimental contamination by E. coli DNA. It is therefore thought that those unmapped ditags represent the DNA fragments that are not included in the current human genome sequences due to the cloning difficulties, or that the DNA fragments that only exist in the Kasumi-1 genome.

### Ditags Identify Missing DNA Fragments

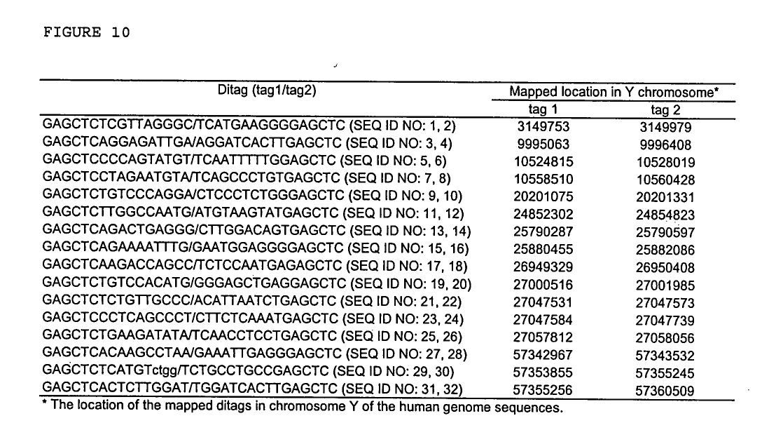

In a preferred embodiment, Ditags can be used to verify if a missed DNA fragment still remains in the genome. The Kasumi-1 cell line was originated from a male, but the whole Y chromosome is not present in the cell line as revealed by Karyotyping (FIG. 4). The mapping results show, however, 16 ditags are mapped specifically to chromosome Y reference ditags (FIG. 10). This information indicates that the detected Y chromosome fragments do not simply disappear from the genome but integrates into other chromosome(s).

### Experimental Verification of Mapping Results

As defined heretofore, a ditag is derived from two ends of a DNA fragment. With 16 bases in each single tag, the two tags readily serve as the sense and antisense PCR primers for the purpose of confirming the presence or absence of the original DNA fragment in the original DNA templates. A positive detection is the indication for the existence of the DNA fragment. A negative detection may imply that the ditag was originated from experimental artifacts, or possibly it is related to the fact that those individual primer sequences are not optimal for PCR amplification.

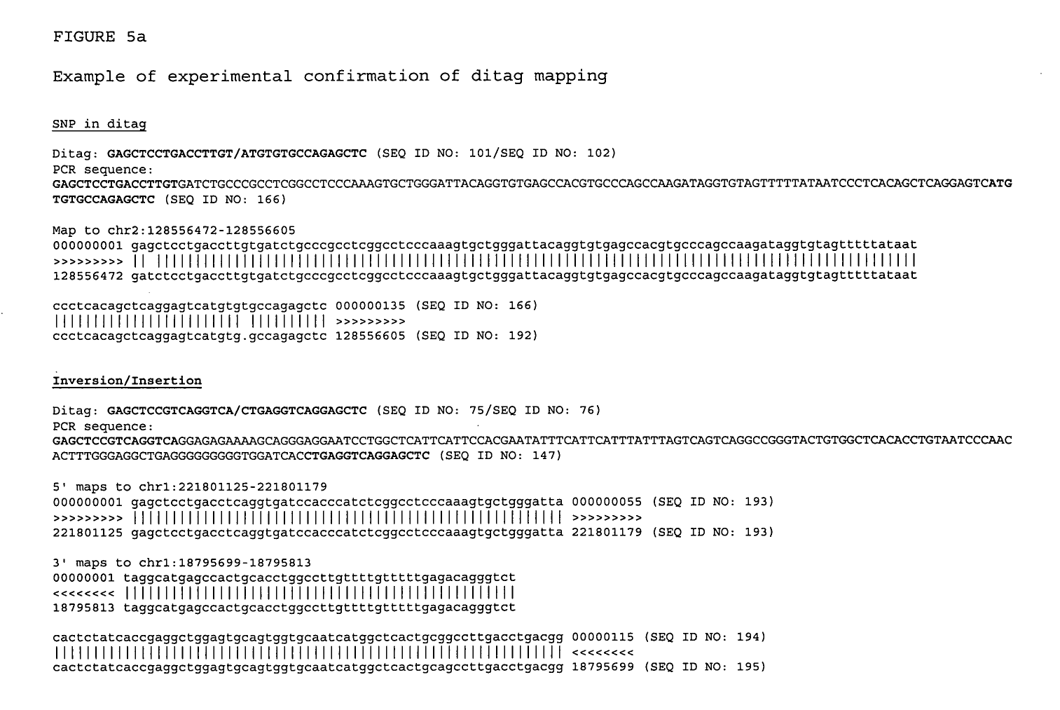

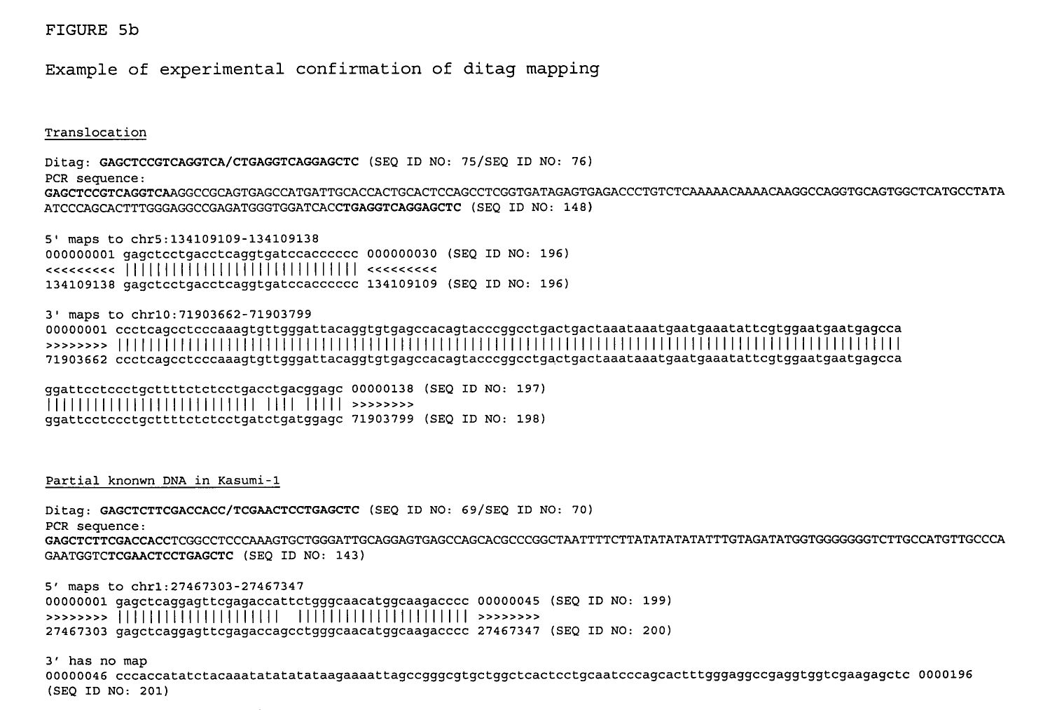

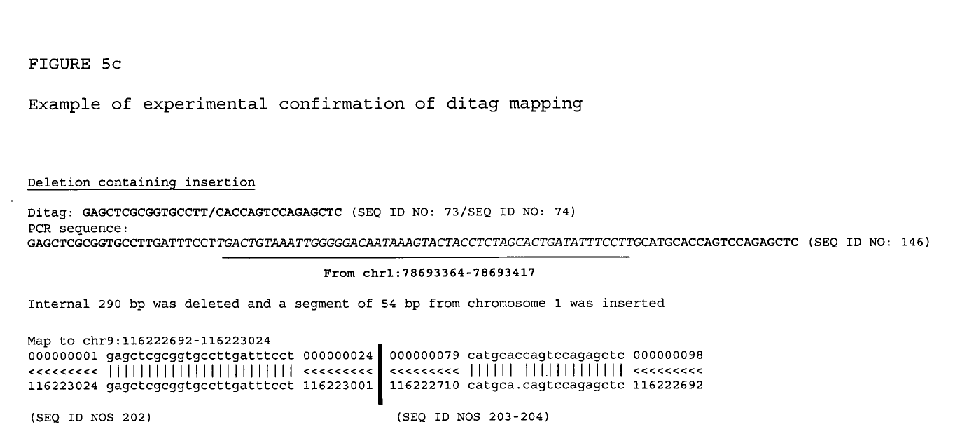

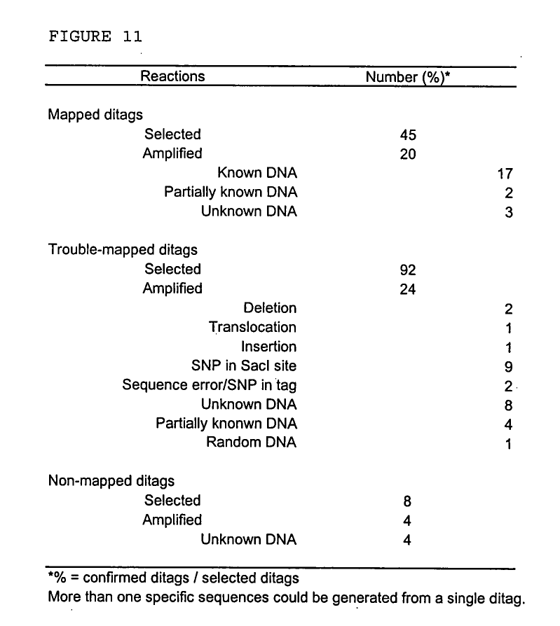

Of the 145 different types of ditags used for the testing, 52 (36%) were experimentally amplified. Mapping the amplified sequences shows that 17 of 20 (85%) of the mapped ditags were from known DNA, and 4 sequences from the non-mapped ditags remain unmapped in the genome. Various origins were identified for the trouble-mapped ditags, including translocation, inversion, insertion and deletion in the Kasumi-1 genome, SNP within the SacI site, SNP/sequencing errors in tag sequences, unknown DNA, DNA partially common between Kasumi-1 genome and the reference human genome, and random DNA sequences not included in the reference human genome sequences (FIGS. 11, 13a-13c, 5).

The DGS system of the present invention provides several unique features for genome analysis including creating new components, such as designing mapping strategy and using PCR for mapping confirmation. The DGS system of the present invention adopts, modifies and integrates multiple components of existing methodologies into a novel linear system. The DGS system includes restriction mapping used for genome analysis, genomic DNA library construction used in conventional DNA cloning, collection of single tags across the genome as used in the Digital Karyotyping technique for detecting genomic copy number changes (18), the fosmid end sequencing used for genome mapping (6), collection of ditags used in the ChIP-PET technology for detecting protein-binding sites in the genome (19), the latest 454 sequencing system for massive sequencing collection (16), and the human genome sequences as the reference for genome study (1).

The integration in the DGS of the present invention makes several significant improvements over the prior art individual components. For example, compared with the single tag used in Digital Karyotyping, the DGS ditag provides a higher specificity for genome mapping, better representation for the changes of deletion, inversion and translocation, and sense and antisense primers for PCR confirmation. By targeting the fragments generated with high frequent restriction enzymes, DGS provides a better resolution than fosmid ending sequencing for genome scanning. DGS differs from ChIP-PET in several aspects: DGS targets well-defined restriction DNA fragments, whereas ChIP-PET uses randomly fractionated DNA fragments; DGS targets DNA fragments across the whole genome, whereas ChIP-PET targets the DNA fragments bound by specific proteins that account for only a small portion of the genome; DGS uses the 454 sequencing system for ditag sequence collection at the genome scale, whereas ChIP-PET uses the conventional DNA sequencing system for ditag sequence collection; the origin of DGS ditags can be easily determined by mapping to a pre-constructed ditag reference database, whereas determining the origin of ChIP-PET ditags in the genome is extremely laborious, as each ditag must be searched across the whole genome sequence since each ChIP-PET ditag is derived from a random DNA fragment.

Each category of DGS ditags of the present invention provides information for its genome origin. The mapped ditags represent the DNA fragments common between the individual genomes and the reference human genome under the defined resolution. The unmapped ditags represent the DNA fragments that are not included in the reference human genome due to cloning difficulties or that only present in the tested genome. The trouble-mapped ditags are also valuable, because they can represent the genomic differences between the tested individual genomes and the reference human genome. However, these ditags can also originate from experimental artifacts. For example, “deletion” might be due to incomplete restriction digestion that leads to the collection of tags downstream of the expected restriction site. Additionally, an “inversion” might be due to the artificial ligation of two fragments in reversed orientation during library construction. A “translocation” might be due to the artificial ligation of two fragments of different chromosomes.

The inventors have discovered that SNP in the SacI restriction site also affects the mapping in which no reference or experimental ditags could be paired. Sequencing error could also affect the mapping results, considering the single-pass nature of the 454 sequences. It is also a challenge for using pure computational approach to definitely define the categories of the structural variation for the trouble-mapped ditags. For example, 14,894 trouble-mapped ditags are grouped under “translocation” i(FIG. 12), although it is unlikely to have so many translocations in the Kasumi-1 genome. One of the causes of the problems is due to the need to separate each trouble-mapped ditag into single tags for further mapping. A single tag has lower specificity than that of a ditag to represent a unique location in the genome. The multiple locations mapped by a single tag create many uncertainties. Therefore, experimental verification for the origins of the trouble-mapped ditags is required to confirm the mapping results. The two tags from DGS ditags can be used as sense and antisense primers for PCR verification. This feature provides an easy means to determine if a ditag was originated from an existing DNA fragment or from an experimental artifact. For the confirmed ditags, the resulting long sequences provide sufficient mapping information for the classification of structural variation.

The DGS system of the present invention cannot cover the entire genome. For example, the mapped ditags in the Kasumi-1 study cover only 24% of the reference ditags in the human genome or 33% of those representing the fragments shorter than 6 kb in the genome. This is due to the limited capacity of 454 sequencing system, and the use of plasmid in DGS as the cloning vector. The current 454 sequencing system provides about 20 Mb capacity per run.

The ditags identified from the sequences provides in the disclosure comprise about less than 1× coverage of the ditags in the genome due to the redundant ditag collection. In an alternate embodiment, the DGS system can use plasmids as the cloning vector in order to detect the shorter DNA fragments for high-resolution scanning. As a result, longer DNA fragments will be excluded for the detection. Those two factors contributed to the negative detection of two ditags from two fusion SacI-SacI DNA fragments originated from chromosomal translocation in Kasumi-1 cell, one ditag represents the 8.9 kb fragment from t(8;21) and the other ditag represents the 4.6 kb fragment from t(21;8) (17, 20). Restricted by probe selection etc, the array-based system also has lower genome coverage, with 25% in the human genome tilling array (11), 39% in Affymatrixs 10 human chromosomes tilling array (12), and 25% in NimbleGen's human genome array (13). Different types of restriction fragments provide difference rate of genome coverage. For example, the SacI fragments of less than 6 kb covers 30% of the human genome, but the rate increases to 47% for the HindIII fragment, and to 60% for the PstI fragments. Following the increased DNA sequencing capacity, it is possible to increase the DGS genome coverage with high-resolution by targeting higher frequent restriction fragments.

Another limitation of DGS system of the present invention is related to detecting genome amplification. Although the copy number of DGS ditags provides potential quantitative information for the detected DNA fragments, it should be cautious to interpret such information as copy number changes in the genome. The DGS process involves multiple library construction propagation and PCR amplification as well. These steps could introduce quantitative changes for the detected ditags. For example, the Kasumi-1 cell contains monosomies (chromosome 13, 16 and X), disomies (chromosome 1, 2, 4, 5, 6, 7, 9, 10, 11. 12, 14, 15, 17, 18, 19, 20 and 21), and trisomies (chromosome 3 and 8). The inventors note that the number of ditags mapped to each chromosome does not parallel the difference of chromosome numbers between monomy, disomy and trisomy chromosomes. Rigorous statistical treatment and real-time PCR is needed to verify if the differences of a ditag copy number do reflect the difference of the corresponding DNA fragments between individual genomes.

### EXAMPLES

### Computational Ditag Analysis

The human genome sequences (NCBI Build 35) were used for the study. Virtual restriction fragments from different restriction sites were generated from the sequences. For each virtual fragment, a 16-bp tag was extracted from its 5′ end and a 16-bp tag from its 3′ end. The two 16-bp tags were then connected to form a virtual ditag to represent its original virtual DNA fragment. The genomic location of the virtual ditags and its original virtual DNA fragment were recorded. The virtual ditags were used for various analyses to determine the correlation between the ditags and the human genome sequences.

### Protocol for DGS Library Construction

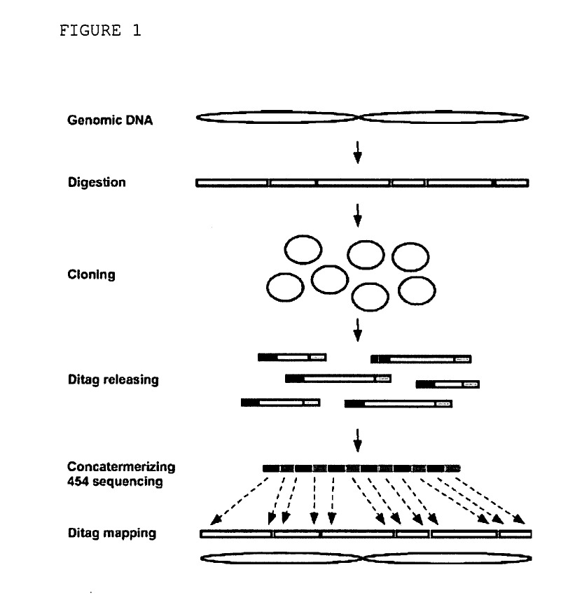

As described in detail below, a genomic DNA sample from the leukemic Kasumi-1 cells was extracted, and fractionated by SacI restriction digestion. The pZEro vector (Invitrogen, Carlsbad, Calif.) was modified in which four wild-type MmeI sites were mutated and two MmeI sites were introduced into the polylinker region next to the SacI site. The SacI-digested DNA sample was cloned into the modified vector to generate a genomic DNA library. The library was then digested by MmeI. The tag-vector-tag fragments were purified from a 1% agarose gel, and re-ligated to form a ditag library. Ditags from the propagated ditag library were released by SacI digestion, purified through a 15% acrylamide gel, and concatemerized by using T4 ligase. The concatermers at 200 to 500 bps were purified from a 5% acrylamide gel and cloned into the p454-SacI vector that contains the 454 adaptor sequences (5′-GCCTCCCTCGCGCCATCAG-3′ (SEQ ID NO: 179), 5′-GCCTTGCCAGCCCGCTCAG -3′ (SEQ ID NO: 180)) to form a ditag concatermer library. After library propagation, the concatermers were released from the library by EcoRI and HindIII digestion and gel purified for 454 DNA sequencing collection (FIG. 1). Ditags were extracted from the 454 sequences based on GAGCTC (SacI site). The ditag data set is stored in http://rulai.cshl.edu/DitagMap/.

The following is the general protocol for DGS library construction of the present invention.

I. Genomic Library (Lib-I) Construction

I-1. DNA Preparation

Purify genomic DNA from cells of choice using QIAamp DNA blood kits (Qiagen), following manufacture's protocol. Measure DNA concentration via Biophotometer (Eppendorf).

Check integrity of genomic DNA by gel electrophoresis. If a portion of DNA is degraded, obtain a new DNA sample and perform the digest again.

I-2.Cleave Genomic DNA with Sac I

Genomic DNA

| | | |
|---|---|---|
| Buffer 1 (10X, NEB) | 25 | μl |
| BSA (100X, NEB) | 2.5 | μl |
| H₂O | to 250 | μl |
| Sac I (20 U/μl, NEB) | 6 | μl |

Aliquot into 50 μl fractions. Incubate at 37° C. for 4 hours or overnight, then add another aliquot of Sac I and continue incubation for another 4 hours. Evaluate digestion by running 5 μl of digest in 1% agarose gel. Follow by extracting with equal volume Phenol/Chloroform.

Precipitate DNA using the following:

| | |
|---|---|
| sample | 250 μl |
| 7.5M NH₄OAc | 125 μl |
| Glycogen (10 μg/μl) | 3 μl |
| 100% EtOH | 850 μl |

Incubate at −20° C. for 15 min, spin for 15 min. Wash twice with 70% ethanol, centrifuge and remove ethanol. Dry the DNA pellet in the air for 10 min. Resuspend in 20 μl LoTE.

I-3. Dephosphorylation of Sac I-Digested Genomic DNA

This step prevents the ligation of digested genomic fragments before clone into vectors. Tests show that dephosphorylation of genomic fragments has little side-effect on the efficiency of library construction.

| | |
|---|---|
| Sac I-digested genomic DNA | 20 μl |
| CIAP buffer (10X, Promega) | 5 μl |
| H₂O | 23 μl |
| CIAP (diluted to 0.1 U/μl, Promega) | 2 μl |

Incubate at 37° C. for 15 min, 56° C. for 15 min. Add a second aliquot of CIAP, and repeat the incubation at both temperatures. Add 150 μl of CIAP stop buffer. Perform Phenol:chloroform extraction and ethanol precipitation. Wash twice with 70% ethanol, centrifuge and remove ethanol. Dry the DNA pellet in the air for 10 min. Resuspend in 20 μl LoTE buffer.

I-4. Clone Genomic DNA Fragments to Vector.

Linearize vector pDGSz that is derived from pZErO-1 (Invitrogen) with only two Mme I sites on each side of the Sac I site.

| | | |
|---|---|---|
| pDGSz (100 ng/μl) | 20 | μl |
| Buffer 1 (10X, NEB) | 5 | μl |
| BSA (100X, NEB) | 0.5 | μl |
| H₂O | 23.5 | μl |
| Sac I (20 U/μl, NEB) | 0.5 | μl |

Incubate at 37° C. for 3 hours. Phenol:chloroform extraction and ethanol precipitation, resuspend pellet in 60 μl LoTE.

| | | |
|---|---|---|
| SacI digested pDGSz | 50 | ng |
| Dephosphorylated genomic DNA | 400 | ng |
| fragments | | |
| 5 x ligase buffer (Invitrogen) | 4 | μl |
| T4 DNA ligase (5 U/ul, Invitrogen) | 1.5 | μl |
| H₂O | to 20 | μl |

Incubate overnight (12-16 hrs) at 16° C. Adjust volume to 200 μl with deionized water, and perform phenol/chloroform extraction and ethanol precipitation (with 3 μl Glycogen). Wash the pellet twice with 70% ethanol, and resuspend in 12 μl water.

I-5. Electroporation

Gently mix 2 μl of the purified ligation DNA with 50 μl electrocompetent cells in pre-chilled 1.5 ml microcentrifuge tubes (Do NOT pipette up and down). Stand on ice for 5 min. Afterward, transfer to pre-chilled BioRad electroporation 0.1 cm cuvettes.

Electroporate cells by BioRad Micropulser with program EC1. The time constant is usually between 4.5 to 5 ms. Add 1 ml room temperature SOC media. Transfer cells to 15 ml Falcon tube. Add another 1 ml SOC in the Falcon tube and shake at 37° C., 250 rpm for 1.5 hrs.

I-6. Library Quality Control

Dilute 10 μl electroporated cells with 100 μl SOC media and plate cells into 90 mm LBzeocin (low-salt LB containing 50 mg/L Zeocin) plate. Incubate overnight at 37° C. Add ⅕ volume of 80% glycerol to the remaining cells and stored at −80° C.

Count the numbers of colonies to estimate clone efficiency. Normally, about 1,000 to about 4,000 colonies can be counted in plates. Using the pZErO-1 derived vector, more than 96% of the colonies contain positive recombinants as revealed by MmeI digestion of plasmids.

I-7. Prepare Lib-I Plasmids

Plate an appropriate volume of the electroporated cells onto large (22×22 cm) agar plates (Zeocin+) (Genetix Q-trays) to grow 200,000˜300,000 colonies per Q-tray. Incubate overnight at 37° C. Scrape colonies with LB media (8-12 ml per Q-tray), and combine all cells in one container. Prepare the LIB-I plasmid by using Promega plasmid Minipreps kit.

The number of colonies required is determined by the desired represented probability of genomic fragment, genome size, average length of insert, and positive recombinant rate, as described by the formula:

N=[ln(1−P)/ln(1−L/G)]/R
N: number of total colonies required
P: desired represented probability of genomic fragment
L: average length of insert
G: size of the genome
R: positive recombinant rate

We routinely target over 1.5×10⁶ colonies as a convenient benchmark for a 90% represented probability of human genomic Sac I fragments.

VIII. Ditag Library (Lib-II) Construction

The vector in Lib-1 contains Mme I sites at both the 5′ and 3′ sides of the genomic DNA insert. Mme I digestion will keep about 20 bp genomic tags at both ends of the vector. Consequently, the tag-vector-tag will be of a constant size (approx. 2843 bp) that can be easily purified through gel electrophoresis.

The 3′-overhang by Mme I digestion can be blunt by T4 DNA polymerase. Ditags will be formed through self-ligation of each tag-vector-tag. The pool of these ditag plasmids becomes the DGS ditag library.

II-1. Digest LIB-I with Mme I

| | | |
|---|---|---|
| LIB-I | 2-5 | μg |
| Buffer 4 (10X, NEB) | 5 | μl |
| SAM (freshly dilute 10X, NEB) | 1 | μl |
| Mme I (2 U/μl, NEB) | 2 | μl |
| H₂O | to 50 | μl |

Incubate at 37° C. from 4 hrs to overnight.

II -2. Recover the Tag-Vector-Tag

Load and run the entire digestion on a 0.7% agarose gel. Excise the 2.8 kb tag-vector-tag bands; purify DNA using the Qiagen agarose gel extraction kit. Quantify the amount of recovered DNA.

II-3. End-Polish the Tag-Vector-Tag Fragments

| | | |
|---|---|---|
| DNA | 200-1,000 | ng |
| 10x Y+/TANGO buffer (Fermentas) | 6.0 | μl |
| 0.1M DTT | 0.3 | μl |
| T4 DNA polymerase (7.9 U/μl, | 0.5 | μl |
| Promega) | | |
| 10 mM dNTP | 0.6 | μl |
| H₂O | to 60 | μl |

Incubate at 37° C. for 5 min, then heat at 75° C. for 10 min. Adjust the volume to 200 μl with deionized water, and perform phenol/chloroform extraction and ethanol precipitation (with 3 μl Glycogen). Wash the pellet twice with 70% ethanol, and resuspend in 60 μl water. Quantify the amount of DNA.

II-4. Form Ditag by Self-Ligation

| | | |
|---|---|---|
| DNA | 100 | ng |
| 5 x ligase buffer (Invitrogen) | 20 | μl |
| H₂O | to 94 | μl |
| T4 DNA ligase (5 U/ul, Invitrogen) | 6 | μl |

Incubate at 16° C. overnight. Adjust the volume to 200 μl with deionized water, and perform phenol/chloroform extraction and ethanol precipitation (with 3 μl Glycogen). Wash the pellet twice with 70% ethanol, and resuspend in 18 μl water.

II-5. Electroporation

Electroporation was accomplished as discussed in step I-5 above.

II-6. Library Quality Control

Dilute 10 μl electroporated cells with 100 μl SOC media and plate cells onto 90 mm LBzeocin plates. Incubate overnight at 37° C. Add 400 μl 80% glycerol to the remaining cells and store at −80° C. Count the numbers of colonies and determine library efficiency. Usually, several thousand colonies can be counted in the 10 μl plating, which indicates the end-polishing and self-ligation were efficient.

Pick 24 colonies for colony PCR with Sp6/T7 or

M13F/M13R primers. Set plasmid pDGSz as control. Run PCR products on a 1.5% agarose gel. Positive ditag clone will show its PCR products are 30-32 bp longer than the control and the rate should be greater than 95%.

II-7. Prepare Lib-II Plasmids

To ensure that the library remains representative of the genomic library (Lib-I), the same or greater number of colonies as in the Lib-I should be obtained. We set the number as 1.8×10⁶. Plate an appropriate volume of the electroporated cells onto LBzeocin Q-trays to get about to about 300,000 colonies per Q-tray.

After overnight (16-18 hrs) 37° C. incubation, scrape the colonies into Solution I (8-12 ml per Q-tray). Combine all cells in one container. Purify LIB-II plasmid DNA by using the Qiagen HiSpeed Plasmid Maxi kit.

IX. Concatermer Library (Lib-III) Construction

1. Release Ditag by Sac I Digestion of LIB-II

| | | |
|---|---|---|
| LIB-II | 300-500 | μg |
| Buffer 1 (10X, NEB) | 100 | μl |
| BSA (100X, NEB) | 10 | μl |
| SacI (20 U/ul, NEB) | 25 | μl |
| H₂O | to 1 | ml |

Aliquot in 100 μl fractions, and incubate at 37° C. overnight. Perform a Phenol/Chloroform extract and ethanol precipitate DNA using the following procedure:

Each 250 μl sample comprises about 125 μl 7.5M NH₄OAc buffer, 10 μl glycogen, and 940 μl 100% EtOH.

Incubate at −80° C. for 30 min, and then spin at 4° C. for 15 min. Wash pellet with cold 70% ethanol, centrifuge and remove ethanol. Dry the DNA pellet on the ice. Resuspend the DNA pellet in 80 μl LoTE buffer.

2. DGS Ditag PAGE Purification

Load 10-15 μl/well of the SacI-digestion DNA into a 2.5% agarose gel and run at 100V for 1 hr. Excise the 29-33 bp DGS ditags band and collect into microspin filter units (SpinX, Costar or Mermaid spin columns, Bio101). Crush the gel slice. Freeze the filter units in either Liquid N₂ (5 min) or dry ice/EtOH (15 min) or −80° C. (20 min). Spin at full speed for 12 min. collect the liquid filtrate. Add 100 μl of LoTE:NH₄OAc (5:1) to each filter unit. Mix and crush the gel slice with pipet tip. Repeat freeze and spin steps. Pool all of the liquid filtrate and measure the final volume.

If the sample volume is greater than 0.3 ml, add 1.5 volumes of 1-butanol, vortex and centrifuge for 2 minutes. Remove butanol (upper) phase and discard. Butanol extraction may be repeated until the sample volume is 0.3 ml or less. Perform ethanol precipitation:

For each 250 μl sample, add 125 μl 7.5M NH₄OAc buffer, 3 μl glycogen, and 940 μl 100% EtOH. Incubate at −80° C. for 30 min, and then spin at 4° C. for 15 min. Wash with cold 70% ethanol, centrifuge and remove ethanol. Dry the DNA pellet on the ice. Resuspend the DNA pellet in 7 μl water.

3. Ditag Concatenation

| | |
|---|---|
| Ditag DNA | 7 μl |
| 5 x ligase buffer (include PEG, | 2 μl |
| Invitrogen) | |
| T4 DNA ligase (5 U/ul) | 1 μl |

Incubate at 16° C. for 30 min. Stop the concatenation reaction by adding 2 μl of standard 6×DNA loading buffer and heating the entire sample at 65° C. for 15 min. Quickly chill the sample on ice.

4. Purify Concatenated Ditags

Load the entire sample into one well of a pre-cast 5% acrylamide Criterion TBE Gel (BioRad), flanked by suitable DNA ladders to allow size determining. Run at 180V for 45 min. Stain with ethidium bromide for 20 min.

Excise the concatenated DNA in two separate fractions: low (150-400 bp, for 454 Life Sciences sequencing) and high (>400 bp, for optional ABI 3730 sequencing). Place the gel slice of each size-fraction into a 1.5 ml microcentrifuge tube. Completely crush the gel slice with pipette tip. Add 200 μl of LoTE:NH₄OAc (5:1) to each tube and elute DNA by heating at 65° C. for 2 hrs.

Use microspin filter units to separate the supernatant (containing the eluted DNA) from the gel pieces by spinning at 13K rpm for 10 min at 4° C. Perform the following ethanol precipitation:

For 200 μl sample, add 100 μl 7.5M NH4OAc buffer, 2 μl glycogen, and 750 μl 100% EtOH. Keep at −20° C. for 20 min, and spin for 15 min. Wash the sample with 75% ethanol. Resuspend the pellet in 6 μl of water.

5. Ligation to p454SacI Vector

The p454SacI was derived from pZErO-1 (Invitrogen) in which two primer sequences from the 454 sequencing system (Primer A 5′-GCCTCCCTCGCGCCATCAG-3′ (SEQ ID NO: 179) and Primer B 5′-GCCTTGCCAGCCCGCTCAG-3′ (SEQ ID NO: 180) were added. Digesting 2 pg of p454Sac plasmid DNA with 10 units of SacI for 2 hours at 37° C. Perform phenol-chloroform extract and ethanol precipitate, resuspend DNA in LoTE at a concentration of 10 ng/μl.

| | |
|---|---|
| SacI digested p454SacI | 1 μl |
| concatenated DNA | 6 μl |
| 5 x ligate buffer (Invitrogen) | 2 μl |
| T4 DNA ligase (5 U/μl, Invitrogen) | 1 μl |

Incubate overnight (12-16 hrs) at 16° C. Add 190 μl water, perform phenol/chloroform extraction and ethanol precipitation (with 1 μl Glycogen). Wash the pellet twice with 70% ethanol, and resuspend in 4 μl water.

6. Electroporation

Same as step I-5.

7. Library QC

Plate 10 μl (out of 2 ml) of electroporated cells onto 90 mm LBzeocin dishes. Incubate overnight at 37° C. Add ⅕ volume of 80% glycerol to the remaining electroporated cells and stored in a −80° C. Count the numbers of colonies.

In order to provide DNA template for 2×10⁵ reads of a typical run of 454 sequencing, more than 1000 colonies should be present in the 10 μl plating.

Pick 24 colonies for colony PCR with Sp6/T7 or M13F/M13R primers. Set plasmid pZErO-1 as the control. Run PCR products in a 1% agarose gel. A positive read for a ditag clone results in PCR products that are 150-400 bp longer than the control with positive rate greater than 95%.

8. 454 Sequencing Collection

To cover for one run of 454 sequencing, at least 2×10⁵ of colonies should be collected. Plate an appropriate volume of the electroporated cells onto LBzeocin Q-trays to grow about 100,000 colonies per Q-tray. Incubation at 37° C. overnight (16-18 hrs), scrape the colonies into Solution I (8-12 ml per Q-tray). Mix all cells in one container.

Prepare LIB-III plasmid DNA by using the Qiagen HiSpeed Plasmid Maxi kit. Release concatenates by Hind III and EcoRI digestion of LIB-III

| | | |
|---|---|---|
| LIB-III | 20 | μg |
| EcoRI buffer (10X, NEB) | 20 | μl |
| Hind III (20 U/ul, NEB) | 2 | μl |
| EcoRI (20 U/ul, NEB) | 2 | μl |
| H₂O | to 200 | μl |

Incubate at 37° C. for 4 hrs to overnight. Perform a Phenol/Chloroform extraction and ethanol precipitate DNA as follows: For 200 μl sample, add 100 μl 7.5M NH₄OAc, 5 μl glycogen, and 750 μl 100% EtOH. Incubate at −20° C. for 20 min, and then spin for 15 min. Wash with 70% ethanol, centrifuge and remove ethanol. Dry the pellet and resuspend in 15 μl water. Load digested products onto a 1.6% agarose gel. Electrophorese at 100V for 55 minutes.

Excise the 200-450 bp fractions and purify DNA using the Qiagen agarose gel extraction kit. For 454 sequencing, at least 100 ng of DNA containing 454 Primer A and Primer B adaptors is required.

Package and ship DNA samples to 454 Life Sciences Company for 454 sequencing collection.

The following are the approximate compositions of the buffers used herein:

CIAP Stop Buffer

10 mM Tris-HCl (pH 7.5)

1 mM EDTA (pH 7.5)

200 mM NaCl

0.5% SDS

LoTE Buffer

3 mM Tris-HCl (pH 7.5)

0.2 mM EDTA

SOC Media (1 L)

20 g tryptone

5 g yeast extract

0.5 g NaCl

2.5 ml 1M KCl

Dissolve in 960 ml of deionized water. Adjust the pH to 7.0 with 5M NaOH. Autoclave and let cool to <55° C. Then aseptically add: 10 ml of sterile 1M MgCl2, 10 ml of sterile 1M MgSO4, and 20 ml of sterile 1M glucose. Store at room temperature.

7.5M NH₄OAc (100 ml) Buffer

57.8 g NH₄OAc dissolved in 70 ml of H₂O at room temperature. Adjust the volume to 100 ml with H₂O. Sterilize the solution by passing it through a 0.22-μm filter, and store in tightly sealed bottles at 4° C. or room temperature.

### Example 2

### Determination of the Genome Origin of Experimental Ditags Through the Ditagmap Reference Database

A reference ditag database named as DitagMap (http://rulai.cshl.edu/DitagMap/) was constructed by using similar process as described in “Computational ditag analysis” except the length of extracted bases from each end was 32 bases. This enabled better mapping of experimental ditags of variable length due to the uncertainty of MmeI digestion. The following protocol below provides a detailed description for mapping experimental ditags to the DitagMap reference database. Based on the mapping situation, ditags were divided into three groups: 1). Mapped ditags, those include the ditags that mapped with reference ditags perfectly and with mismatches up to two bases, of which the p values are higher than the cutoff of 1.0e-5; 2) Trouble-mapped ditags, those are the ditags of which the combined p values of mapping two single tags in reference ditag database are higher than the cutoff of 1.0e-3, or, any single tag mapping p value is larger than 1.0e-3, which allows at most one mismatch with reference tags; and 3) Unmapped ditags, these are the ditags of which the p values are less than the cutoff 1.0e-3 when their two single tags are mapped to reference ditag database.

Construction of reference ditag database for Example 4. A reference ditag database was constructed to determine the genome origin of experimental ditags in Example 4. This database contains reference ditags extracted from virtual DNA sequences of the following sources (5′ 17 bases-17 bases 3′): the human genome reference sequences HG18:

http://hgdownload.cse.ucsc.edu/goldenPath/hg18/bigZips/; human dbSNP 126:
http://www.ncbi.nlm.nih.gov/SNP/)ftp://ftp.ncbi.nih.gov/snp/organisms/human_{—}9606/; chimpanzee genome reference sequences: PanTro2, March 2006,
http://hgdownload.cse.ucsc.edu/goldenPath/panTro2/bigZips/; the fosmid pairing end sequences:
http://www.ncbi.nlm.nih.gov/Traces/trace.cgi?&cmd=retrieve&val=CENTER_PROJECT%20%3D%20%22G248%22&size=0&retrieve=Submit; Celera genome sequences:
http://www.ncbi.nlm.nih.gov/sites/entrez?db=genomeprj&cmd=Link&LinkName=genomeprj_nuccore_wgs&from_uid=1431; Venter genome sequences:
ftp://ftp.ncbi.nih.gov/pub/TraceDB/Personal_Genomics/Venter/; Watson raw 454 genome sequences:
http://ftp.ncbi.nih.gov/pub/TraceDB/Personal_Genomics/Watson/.

A MySQL-based database was constructed for ditag analysis, including extracting ditags from raw 454 sequences, mapping the ditags to the reference ditags, and outputting the mapping results.

Each experimental ditag in Example 4 was mapped to the reference ditag database. Based on the mapping result, a ditag is classified into two subgroups. 1). Mapped ditags. These includes the ditags that mapped to reference ditags perfectly, or with one-base mismatches in each single tag to compromise potential sequencing error or SNP; 2) Trouble-mapped ditags. These include the ditags whose both single tag maps to unexpected locations, whose only one single tag maps or whose both single tags do not map to any reference ditags.

Experimental verification of the results of Example 4 was derived as follows. In brief, each single tag of 16 bases in ditag sequences was used to design a sense primer and an antisense (reverse/complementary) primer, with four extra bases (ATTC) added to the 5′ end of sense primer and TTAG to the 3′ end of the antisense primer to increase the primer length to 20 bps. PCR was performed for 30 cycles at 950 C 30 sec, 600 C 30 sec, and 720 C 60 sec. PCR products were checked on 2% agarose gels, or cloned into the pGEM-T vector (Promega) for sequencing confirmation. The resulting sequence was mapped to the human genome sequences through the UCSC genome browser (http://genome.ucsc.edu/cgi-bin/hgGateway). To determine if the genome variations detected by ditags are present in different individual genomes, a Coriell human DNA panel [Human Variation Panel-Caribbean that includes 8 Caucasian and 2 Black (GM17350GM17359)] were used as the templates (http://ccr.coriell.org/nigms/nigms_cgi/panel.cgi?id=2&query=HDPCARIB).

The mapping of the ditags in Examples 1-3 was accomplished as follows. A reference ditag contains a 32-bp tag from the 5′ end and a 32-bp tag from the 3′ end of a virtual DNA fragment. Each experimental ditag is searched in the DitagMap reference database. Experimental ditags shorter than 32 bps are compared with reference ditags of the same length, and the total mismatches are counted without allowing gaps. Ditags longer than 31 bps are compared with reference ditags with extra bases: the 16-bps in both ends are aligned with the ends of each reference ditag; the extra bases between the two 16-bp are compared with the bases in the reference ditag. Those with matches are assigned to the corresponding single tag. Because the experimental ditags can be cloned in either forward or reverse orientation, the mapping process is also performed through reverse/complement of each experimental ditag. The length of experimental ditag and the mismatches with each reference ditag are used to calculate the p-score (see below).

The process for mapping a single tag is used for the trouble-mapping ditags. The left and right single tags of these ditags are separately mapped with single tags of all reference ditags. The alignment is extended to the 3′-end of the single tag until a mismatch is detected. The same process is performed through the reverse complement of each single tag. When the two single tags of an experimental ditag map to different reference ditags, combined p value will be calculated by counting the total mismatches in the alignment between the ditag and the mapped reference ditags. If the combined p value is lower than the cutoff of 1.0e-3, this ditag is considered as not mapped in both ends, and the p value will be calculated for each single tag based on the mismatches in its 16-bp terminal region. Based on the definition of genomic structural variation, these ditags are further classified into the different types of variation. In the case of multiple genomic locations and multiple types of variation assigned for one experimental ditag, all locations and variation types will be reported.

A p-score calculation is made. A p-score is used to describe the probability of a ditag or a single tag to be mapped in the whole ditag reference database. Considering the potential mismatches caused by sequencing error and/or SNPs, the probability of an experimental ditag/tag (w_{ob}) mapping in the reference database is calculated by using the formula: p-score=

$$1 - {\prod }_{{w}_{i} ∈ R} ( 1 - p ( {w}_{ob} , {w}_{i} ) )$$

$$while$$

$$p ( {w}_{ob} , {w}_{i} ) = {p}_{err}^{m} * {( 1 - {p}_{err} )}^{( L - m )}$$

R′ represents the whole set of ditags/tags obtained from the reference human genome, wᵢ represents the ith ditag/tag in it. L is the length of the experimental ditag or single tag, with length of perfect match or one-base mismatch. m is the number of the mismatched base(s) between w_{ob} and wᵢ. pₑᵣᵣ is the rate of sequencing error and/or SNPs.

A ditag is considered being mapped in the reference ditag database, if the p value is higher than the cutoff of 1.0e-5. The setting of the cutoff at 1.0e-5 is based on the effects of sequencing error, SNP, and multiple tests for hundred thousand experimental ditags. Using this cutoff, 0-2 mismatches between experimental ditag and reference ditag are allowed. If the p value is lower than 1.0e-5, a combined p value for each ditag or for single tag will be calculated. If the combine p value or single tag mapping p value is higher than cutoff of 1.0e-3, this ditag/tag is considered as trouble-mapped; otherwise, this ditag/tag is regarded as non-mapping in the reference database.

### Example 3

The protocol in the following paragraphs provides a detailed description for the process. In brief, each single tag of 16 bases in ditag sequences was used to design a sense primer and an antisense (reverse/complementary) primer. The original SacI-digested DNA sample was used as the template. PCR was performed at 30 cycles at 95° C. 30 sec, 58° C. 30 sec, and 72° C. 80 sec. PCR products were cloned into the pGEMT vector and sequenced by using the T7 primer. The longer sequences were sequenced from the other end by using the SP6 primer. A qualified sequence should contain the sense and the antisense primer sequences at the two ends. Each sequence was mapped to the human genome sequences through the UCSC genome browser (http://www.genome.ucsc.edu/).

### Protocol for PCR Verification of Ditag Mapping Result

In the following process, each single tag in a ditag is used as a sense and an antisense primer, the original DNA used for ditag collection is used as the template for PCR amplification. The PCR product is cloned, sequenced and mapped to the genome to verify its genome origin. The whole process can be scaled up to 96× format for high-throughput analysis.

1. Digest Genomic DNA with Sac I

| | |
|---|---|
| Genomic DNA (100 ng/μl) | 60 μl |
| Buffer 1 (10X, NEB) | 20 μl |
| BSA (100X, NEB) | 2 μl |
| Sac I (20,000 u/μl, BioLabs) | 4 μl |
| ddH₂O | 114 μl |

Incubate at 37° C. for 3 hours. Evaluate the digestion by running 2 μl of DNA on a 1% agarose gel.

2. PCR Amplification

| | 1x | 100x |
|---|---|---|
| Digested DNA | 0.1 μl/well | 10 μl |
| template | | |
| 10X Ramp-Taq | 3.5 μl/well | 350 μl |
| Buffer | | |
| MgCl₂ (50 mM) | 1.5 μl/well | 150 μl |
| dNTPs (2.5 mM) | 1 μl/well | 100 μl |
| Taq polymerase (5 | 0.2 μl/well | 20 μl |
| u/μl) | | |
| Sense primer | 1 μl/well | — |
| (10 mM) | | |
| Antisense primer | 1 μl/well | — |
| (10 mM) | | |
| ddH₂O | 26.7 μl/well | 2670 μl |

Aliquot 33 μl of the mixture per each well containing sense and antisense primers, and set the PCR conditions as follows:

| | | Number of |
|---|---|---|
| Temperature | Time | cycles |
| 95° C. | 6 min 30 sec | 1 |
| 94° C. | 30 sec | 30 |
| 60° C. | 30 sec | |
| 72° C. | 1 min 20 sec | |
| 72° C. | 5 min | 1 |
| 4° C. | ∞ | |

3. Purify PCR Products

| | | |
|---|---|---|
| PCR products | 35 | μl/well |
| CH₃COONH₃ (7.5 M) | 10 | μl/well |
| Glycogen (20 mg/ml) | 1 | μl/well |
| Ethanol | 100 | μl/well |

Mix the samples well and store at −20° C. for 10 minutes. Centrifuge samples at 4,000 rpm at 4° C. for 30 minutes. Pour out supernatant from the plate. Wash with 90 μl/well 70% ethanol, and centrifuge at 4000 rpm for 15 min. Pour out supernatant from the plate, and centrifuge at 250 rpm for 1 min. Air dry pellets for 10 min. Resuspend pellets with 5 μl/well ddH₂O.

4. Clone PCR Products

| | 1x | | 100x |
|---|---|---|---|
| PCR products | 2 | μl/well | — |
| pGEM-T (50 ng/μl) | 0.2 | μl/well | 20 μl |
| T4 DNA ligase | 0.15 | μl/well | 15 μl |
| (3 u/μl) | | | |
| 2X ligase buffer | 2.5 | μl/well | 250 μl |
| ddH₂O | 0.15 | μl/well | 15 μl |

Aliquot 3 μl/well containing 2 ul PCR products. Centrifuge the plate at 1,400 rpm for 1 min, set the ligation at 4° C. overnight.

5. Transformation

Add 2 μl/well of ligation into 25 μl/well of Top10 competent cells. Mix gently and keep on ice for 25 min. Transfer the plate to 42° C. water bath for 50 sec. Keep the plate on ice for 2 min, add 80 μl/well of SOC. Shake the plate at 250 rpm at 37° C. for 1 hr. Transfer all the cell solution to each unit of Q-tray containing ampicillin LB. Add 10 μl X-gal in each unit. Spread cells by using beads (Genetix). Incubate cells at 37° C. for 14-16 hrs.

6. Colony PCR

Prepare PCR mixture as follows:

| | | |
|---|---|---|
| 10 X PCR Buffer | 3,000 | μl |
| MgCl₂ (50 mM) | 1,600 | μl |
| DMSO | 1,400 | μl |
| dNTP (2.5 mM) | 600 | μl |
| T7 Primer (10 μM) | 240 | μl |
| SP6 Primer (10 μM) | 240 | μl |
| ddH₂O | 22,000 | μl |

and store the solution at −20° C. Before use, add taq polymerase in the following concentrations:

| | 1x | 100x |
|---|---|---|
| Mixture | 8 μl/well | 800 μl |
| Taq | 0.1 μl/well | 10 μl |
| polymerase | | |

and aliquot 8 μl of the mixture/well. Dip individual colony/well by using pipette tip, and perform PCR with the following conditions:

| Temperature | Time | | Number of cycles |
|---|---|---|---|
| 95° C. | 7 | min | 1 |
| \| | 30 | sec | 20 |
| 94° C. | | | |
| 55° C. | 30 | sec | |
| 72° C. | 1 | min | |
| | 20 | sec | |
| 72° C. | 2 | min | 1 |

| | |
|---|---|
| 4° C. | ∞ |

while adding 50 μl of ddH₂O to each well to dilute PCR products.

7. Sequencing Reaction

| | 1x | 100x |
|---|---|---|
| Diluted PCR products | 1 μl/well | — |
| 5X Big dye sequencing | 1.6 μl/well | 160 μl |
| buffer | | |
| Big dye | 0.2 μl/well | 20 μl |
| T7 or SP6 primer | 0.2 μl/well | 20 μl |
| (10 mM) | | |
| ddH₂O | 5 μl/well | 500 μl |

The sequencing conditions are set as follows:

| Temperature | Time | | Number of cycles |
|---|---|---|---|
| \| | 1 | min | 1 |
| 96° C. | | | |
| 96° C. | 10 | sec | 50 |
| 50° C. | 5 | sec | |
| 60° C. | 3 | min | |
| | 30 | sec | |
| 4° C. | 1 | min | 1 |

| | |
|---|---|
| 16° C. | ∞ |

It should be understood by those of ordinary skill that the sequencing reaction for long fragments must be performed with T7 and SP6 primer separately, in order to obtain longer DNA sequences from both ends until the fragment is fully covered.

8. Purify Sequencing Products

| | 1x | 100x |
|---|---|---|
| EDTA (0.125M, pH8.0) | 2 μl/well | 200 μl |
| Ethanol | 30 μl/well | 3,000 μl |

32 μl solution of the above solution are added to each well containing sequencing products, and are kept at room temperature for 15 min. Centrifuge samples at 4,000 rpm at 4° C. for 30 min. Pour out supernatant from the plate and centrifuge at 250 rpm for 1 min. Add 60 μl 70% ethanol/well, and centrifuge at 4000 rpm for 15 min. Pour out supernatant from the plate, centrifuge at 250 rpm for 1 min. Air dry the samples. Add 7 μl/well formamide, and store samples at room temperature for 1 hr. Heat the plate at 95° C. for 3 min and move it on ice for 2 min. Centrifuge the plate at 1400 rpm for 1 min. Load the plate in a ABI 3730xl DNA sequencer to collect DNA sequences.

### Example 4

### Ditag Mapping of Three Sets of Human Genome Sequences

Using the human genome reference sequences HG18 as a model, we studied the feasibility of using the 454 system for ditag sequence collection and characterized the relationship between ditag and genome structure.

We analyzed various types of virtual restriction fragments in HG18 to find the range of the total bases from the corresponding ditags. The result shows that the total number of ditag-derived bases from the 6-base restriction fragments is between 2 to 45 Mbs (Table 1), a range that matches the capacity of the 454 sequencing system per run. The total bases from 8-base restriction fragments is far lower than the range, whereas the 4base restriction fragments are far higher than this range (data not shown). Therefore, the 6-base restriction fragments are the suitable choice.

TABLE 1
Number of fragments and ditag bases by 6-base restriction in HG18

| Restriction | Restriction | Total | Total bases |
|---|---|---|---|
| enzymes | sites | fragments/ditags | from ditags* |
| PstI | CTGCAG | 1,306,835 | 44,432,390 |
| NsiI | ATGCAT | 928,031 | 31,553,054 |
| HindIII | AAGCTT | 842,432 | 28,642,688 |
| XbaI | TCTAGA | 804,875 | 27,365,750 |
| EcoRI | GAATTC | 783,915 | 26,653,110 |
| BglII | AGATCT | 775,788 | 26,376,792 |
| SacI | GAGCTC | 599,852 | 20,394,968 |
| SphI | GCATGC | 549,919 | 18,697,246 |
| ScaI | AGTACT | 543,087 | 18,464,958 |
| ApaI | GGGCCC | 462,363 | 15,720,342 |
| EcoRV | GATATC | 433,575 | 14,741,550 |
| SpeI | ACTAGT | 395,746 | 13,455,364 |
| BamI | GGATCC | 350,470 | 11,915,980 |
| KpnI | GGTACC | 288,593 | 9,812,162 |
| XhoI | CTCGAG | 121,323 | 4,124,982 |
| Asp130I | ATCGAT | 85,897 | 2,920,498 |

*Seventeen bases from each end of a fragment were used for the calculation.

The size of the restriction DNA fragments represents the resolution of the detection. To investigate at what resolution the ditags can provide, we analyzed the size distribution of virtual 6-base restriction fragments in HG18. The result shows that the size distribution varies widely, depending on the type of restriction fragments. For example, the total number of Asp1301 (ATCGAT) fragments is 84,919 but the number increases to 1,290,483 for the PstI (CTGCAG) fragments. The difference is mainly due to the changes in the number of smaller fragments.

Setting 6 kb as the cut-off. The number of fragments shorter than 6 kb between Asp130I fragments and PstI fragments varies over 75 folds (15,695 for Asp130I fragments verses 1,182,877 for PstI fragments). In contrast, the number of the fragments longer than 6 kb is rather constant between different types of restriction fragments, i.e., less than 2-fold changes is present between Asp1301 and PstI fragments (Table 2).

TABLE 2
Length of 6-base restriction fragments in HG18

| | | | Fragments <= 6 kb |
|---|---|---|---|
| Enzymes | Total fragments | Fragments > 6 kb (%) | (%) |
| PstI | 1,306,835 | 137,552 (11) | 1,169,283 (89) |
| NsiI | 928,031 | 167,257 (18) | 760,774 (82) |
| HindIII | 842,432 | 153,346 (18) | 689,086 (82) |
| XbaI | 804,875 | 160,840 (20) | 644,035 (80) |
| EcoRI | 783,915 | 161,487 (21) | 622,428 (79) |
| BglII | 775,788 | 160,909 (21) | 614,879 (79) |
| SacI | 599,852 | 171,436 (29) | 428,416 (71) |
| SphI | 549,919 | 180,263 (33) | 369,656 (67) |
| ScaI | 543,087 | 174,386 (32) | 368,701 (68) |
| ApaI | 462,363 | 144,722 (31) | 317,641 (69) |
| EcoRV | 433,575 | 170,269 (39) | 263,306 (61) |
| SpeI | 395,746 | 169,476 (43) | 226,270 (57) |
| BamI | 350,470 | 152,405 (43) | 198,065 (57) |
| KpnI | 288,593 | 151,244 (52) | 137,349 (48) |
| XhoI | 121,323 | 87,780 (72) | 33,543 (28) |
| Asp130I | 85,897 | 70,443 (82) | 15,454 (18) |

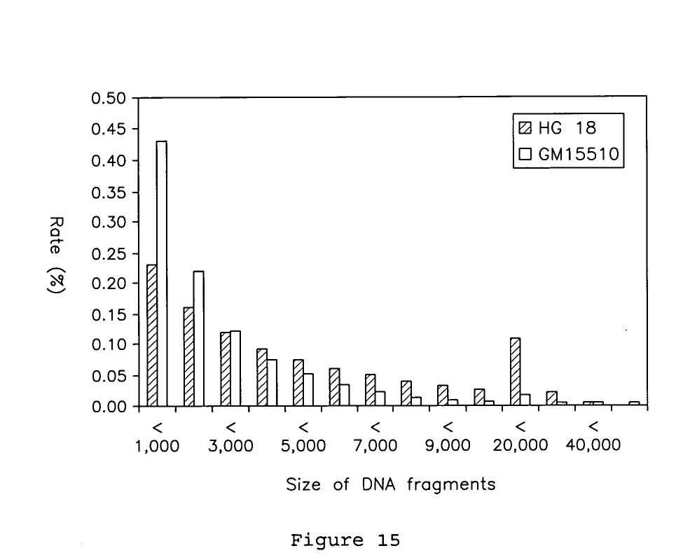

Although the absolute number of the longer fragments remains rather stable in different types of 6-bp restriction fragments, the proportion decreases substantially in higher frequent restriction fragments. This information indicates that the resolution of detection can be pre-determined by selecting different types of 6base restriction fragments. For example, of the 593,142 SacI fragments, 72% are shorter than 6 kb and 23% are shorter than 1 kb (FIG. 15). By targeting higher frequent restriction fragments, higher resolution and higher genome coverage can be reached.

Ditags have short sequences (on average 34 bp per ditag), and we sought to determine whether the ditag population is highly specific in representing their original DNA fragments at the genome level. Our study shows that this is the case indeed. Taking the ditags from SacI fragments as an example, there are 593,142 SacI fragments in HG18. Of the ditags extracted from these fragments, 95% (565,472) map back specifically to their original fragments. The high specificity is consistent across different chromosomes except chromosome Y due to its repetitive sequence nature (Table 3A).

TABLE 3
Specificity of SacI ditags in the human genome
A. Ditag specificity*

| | | | Specific ditags |
|---|---|---|---|
| Choromosome | Total ditags | Non-specific ditags (%) | (%) |
| 1 | 50,228 | 2,502 (5) | 47,726 (95) |
| 2 | 47,985 | 1,727 (4) | 46,258 (96) |
| 3 | 37,363 | 1,142 (3) | 36,221 (97) |
| 4 | 32,682 | 1,430 (4) | 31,252 (96) |
| 5 | 34,445 | 2,107 (6) | 32,338 (94) |
| 6 | 34,938 | 4,458 (13) | 30,480 (87) |
| 7 | 31,806 | 1,855 (6) | 29,951 (94) |
| 8 | 28,929 | 1,215 (4) | 27,714 (96) |
| 9 | 25,537 | 2,158 (8) | 23,379 (92) |
| 10 | 29,252 | 1,567 (5) | 27,685 (95) |
| 11 | 30,346 | 1,316 (4) | 29,030 (96) |
| 12 | 26,467 | 1,053 (4) | 25,414 (96) |
| 13 | 16,726 | 484 (3) | 16,242 (97) |
| 14 | 18,386 | 672 (4) | 17,714 (96) |
| 15 | 18,863 | 1,384 (7) | 17,479 (93) |
| 16 | 20,214 | 1,394 (7) | 18,820 (93) |
| 17 | 20,500 | 1,305 (6) | 19,195 (94) |
| 18 | 14,479 | 363 (3) | 14,116 (97) |
| 19 | 15,038 | 837 (6) | 14,201 (94) |
| 20 | 15,206 | 328 (2) | 14,878 (98) |
| 21 | 7,207 | 314 (4) | 6,893 (96) |
| 22 | 10,391 | 574 (6) | 9,817 (94) |
| X | 28,420 | 2,568 (9) | 25,852 (91) |
| Y | 4,397 | 1,580 (36) | 2,817 (64) |
| Total | 599,805 | 34,360 | 565,472 (94) |

*A specific ditag refers to a ditag that exists only ionce in the whole genome.

Furthermore, the high specificity is not only for the ditags from the non-repetitive sequences but also for the ditags from the repetitive sequences. Half of the human genome is composed of repetitive DNA. Reflecting this nature, 27% of ditags are from the purely repetitive DNA fragments and 40% of ditags are from the fragments across the non-repetitive and the repetitive DNA (in a ditag, one single tag is from the non-repetitive region and the other is from the repetitive region). For the ditags from the purely repetitive DNA fragments, 89% remain specific; for the ditags across the repetitive and non-repetitive regions, 98% are specific (Table 3B). The high specificity of ditags for the repetitive DNA fragments enables use of ditag to analyze the structure in the repetitive regions of the genome.

TABLE 3B
B. Ditags from non-repetitive and repetitive regions*

| Genomic region | | | |
|---|---|---|---|
| Tag1 | Tag2 | Number of ditags | Specific ditags |
| Repetitive | Repetitive | 159,794 (27) | 141,259 (88) |
| Repetitive | Non-repetitive | 119,256 (20) | 115,627 (97) |
| Non-repetitive | Repetitive | 119,278 (20) | 115,705 (97) |
| Non-repetitive | Non-repetitive | 201,477 (34) | 192,881 (96) |
| Total | | 599,805 (100) | 565,472 (94) |

*“Repetitive” region refers the sequences covered by RepeatMasker program.

To evaluate DGS experimentally, we collected ditags from GM15510 DNA. The same DNA was used for the construction of a fosmid library. This library was pair-end sequenced extensively, with the collection of 1.7 Gb, or more than half of human genome contents (International Human Genome Study Consortium. 2004). These sequences were used for studying genome variation with the identification of 297 variations in the GM15510 genome that are different from the human genome reference sequences (Tuzun et al. 2005). By collecting ditags from the same DNA sample, the existing rich genomic information provides a control to evaluate DGS for detecting genome structural changes.

We analyzed two types of restriction fragments from GM15510 DNA: the SacI fragment that has a modest restriction frequency, and the HindIII fragment that has higher restriction frequency (Table 2). By using one 454 GS20 sequencing run, we collected 160,537 raw sequences of 14 Mb from SacI ditag and HindIII ditags. From those sequences, we identified 331,010 ditag copies and 81,890 unique ditags including 46,354 SacI ditags and 35,536 HindIII ditags (Table 4, FIG. 12).

TABLE 4
Mapping summary for the ditags collected from GM15510 DNA

| Items | SacI | HindIII | Total |
|---|---|---|---|
| Total bases | 8,144,009 | 6,380,307 | 14,524,316 |
| Total sequences | 89,352 | 71,185 | 160,537 |
| Total ditags identified | 280,487 | 260,359 | 540,846 |
| Total unique ditags | 46,354 (100) | 35,536 (100) | 81,890 (100) |
| Mapped ditags | 40,985 (88.4) | 29,964 (84.3) | 70,949 (86.6) |
| Human genome | 40,380 (87.1) | 29,447 (82.9) | 69,827 (85.3) |
| sequences (HG18) | | | |
| Perfect match | 37,318 (80.5) | 26,564 (74.8) | 63,882 (78.0) |
| 1-base mismatch | 2,134 (4.6) | 1,850 (5.2) | 3,966 (4.8) |
| SNP | 166 (0.4) | 83 (0.2) | 249 (0.3) |
| Homopolymer | 772 (1.7) | 958 (2.7) | 1,730 (2.1) |
| Chimpanzee genome | 277 (0.6) | 181 (0.5) | 458 (0.6) |
| sequences | | | |
| Human genome | 318 (0.7) | 328 (0.9) | 664 (0.8) |
| variations* | | | |
| GM15510 fosmid | 25 | 30 | 55 |
| sequences | | | |
| Celera human genome | 167 | 147 | 314 |
| sequences | | | |
| Venter genome | 269 | 274 | 543 |
| sequences | | | |
| Watson genome | 28 | 34 | 62 |
| sequences | | | |
| Trouble mapped ditags | 5,248 (11.6) | 5,533 (15.7) | 10,781 (13.3) |
| Two single tags | 3,509 (7.6) | 4,549 (12.8) | 8,058 (9.8) |
| mapped | | | |
| Same chromosome | 1,073 (2.3) | 2,091 (5.9) | 3,164 (3.9) |
| Different | 2,436 (5.3) | 2,458 (6.9) | 4,894 (6.0) |
| chromosomes | | | |
| Only one single tag | 1,739 (3.8) | 984 (2.8) | 2,723 (3.3) |
| mapped | | | |
| Both single tags don't | 121 (0.3) | 39 (0.1) | 160 (0.2) |
| map | | | |

*The 664 ditags map to 1,007 loci across diffeent genomes.
The ditag mapped to more than one individual genome was counted only once.

The genome coverage is about 10% for SacI ditags and 5% for HindIII ditags when referring to the fragments <6 kb that are clonable by plasmid vector, or 8% for SacI ditags and 4% for HindIII ditags when referring to all fragments of the genome (Table 2). The ratio between the total collected ditag copies and the total unique ditags is about 4 to 1. In general, the results between SacI and HIndIII data collections are consistent.

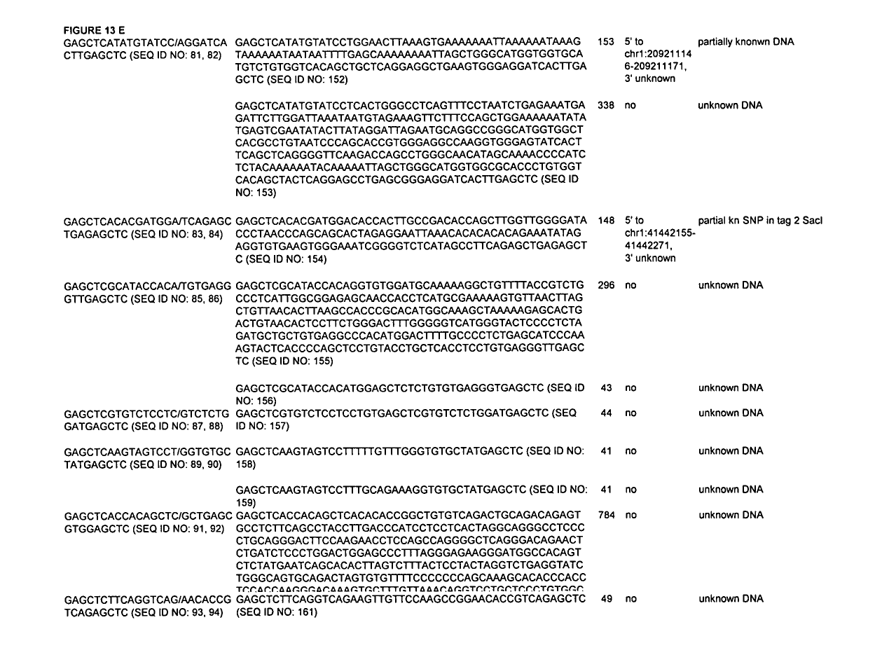

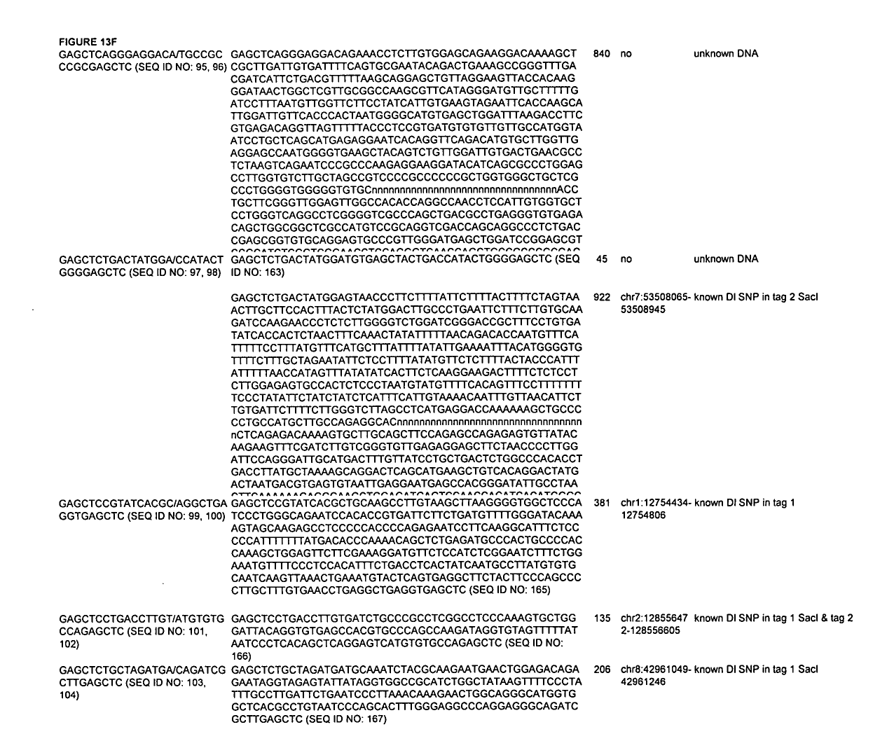

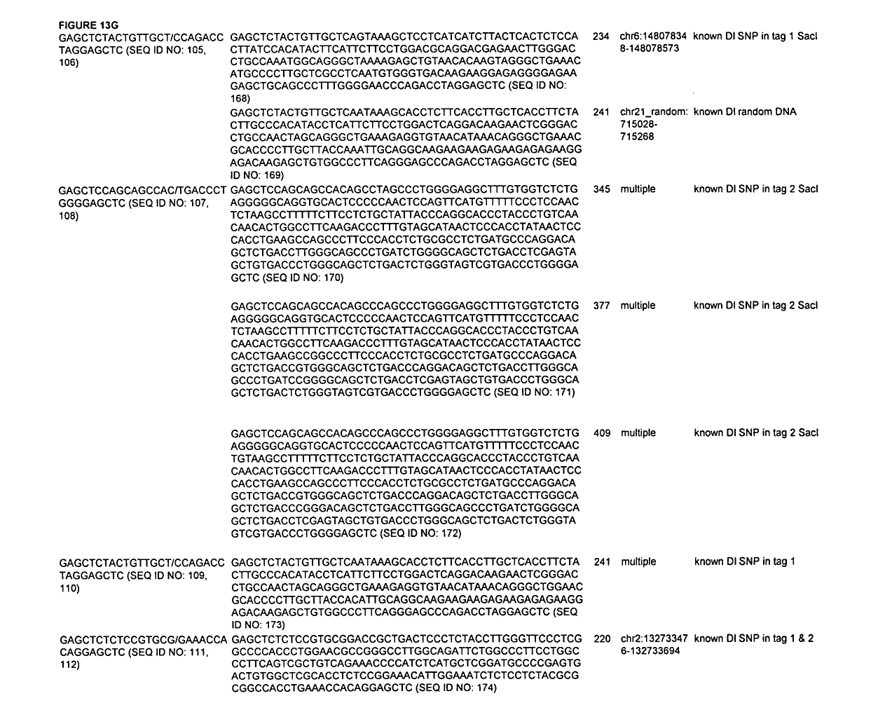

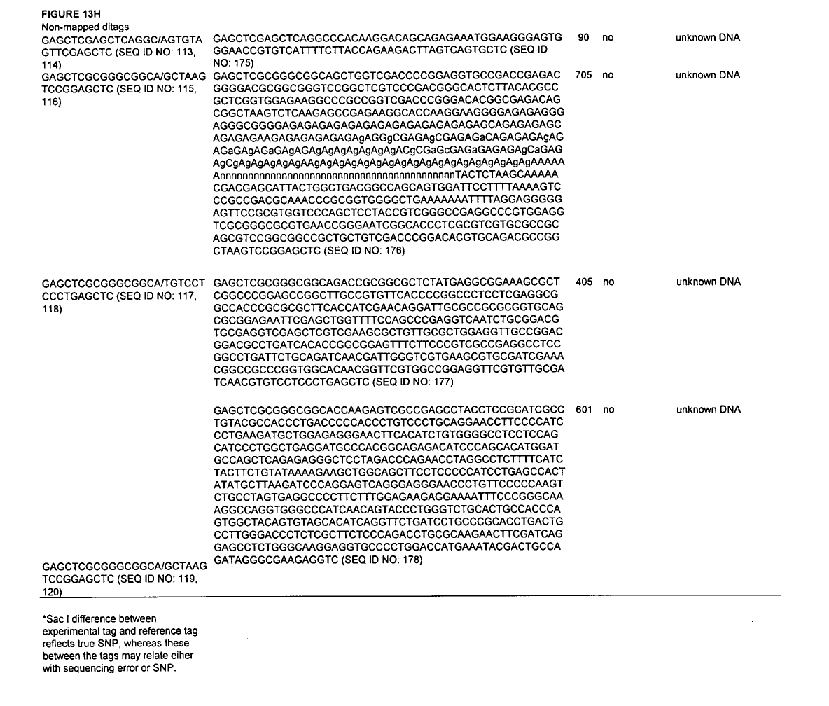

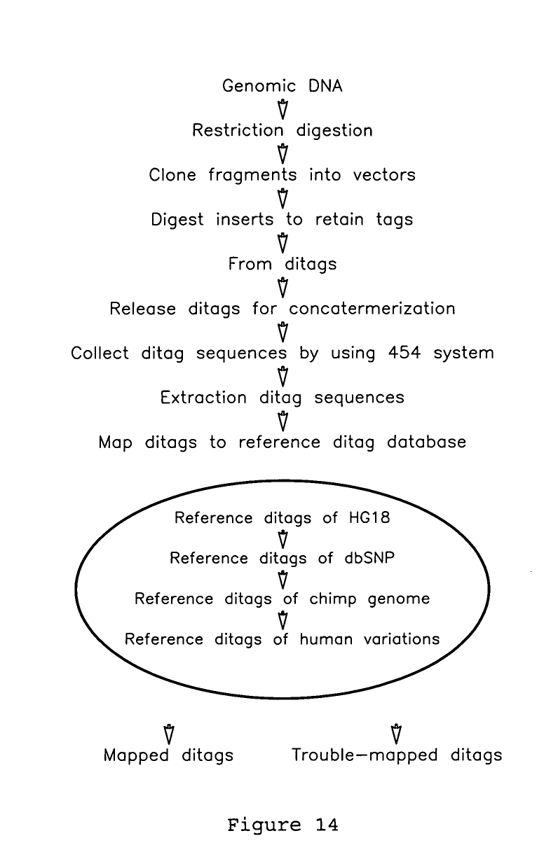

In order to determine the genome origin of the detected ditags, we developed a comprehensive reference ditag database. This database contains virtual ditags extracted from virtual restriction fragments in HG18. In addition, the database also includes reference ditags containing known SNP to identify the experimental ditags containing SNP. Taking advantage of the high sequence similarity between the human genome and the chimpanzee genome (Li and Saunders 2005), reference ditags were also extracted from the chimpanzee genome reference sequences to identify the ditag whose original fragment is not included in the human genome reference sequences but whose homologous counterpart is present in the chimpanzee genome sequences. To identify the ditags from the variations determined by the GM15510-derived fosmid pair-end sequencing, the reference database includes the reference ditags extracted from the sequences of these variations. To identify the ditags from the variations in the available individual human genome sequences, ditags were also extracted from the assembled Celera human genome sequences, the unassembled Venter genome sequences and the unassembled Watson 454 genome sequences. FIG. 13 summarizes the reference ditag information.

The experimental ditags were mapped to the reference ditags of HG18. For the ditags without mapping, allowing one-base mismatch in each single tag between the experimental ditag and the reference ditag identified the ditags containing potential sequencing error or SNP. Considering that the 454 sequencing has difficulty in determining the precise number of homo-bases in the homopolymer region (Goldberg et al. 2006), the ditags with homopolymer-bases were identified, and mapped to the reference ditags by allowing multiple mis-matches for the homo-bases (Ng et al. 2006). Through these processes, 78% of ditags were identified as the perfect mapped ditags, 0.3% as SNP-containing ditags and 5% as the ditags from sequencing errors or unknown SNP, and 2% as homopolymer ditags. In total, 85.3% of ditags from the GM15510 genome maps to the human genome reference sequences HG18 (Table 4).

TABLE 4
Mapping summary for the ditags collected from GM15510 DNA

| Items | SacI | HindIII | Total |
|---|---|---|---|
| Total bases | 8,144,009 | 6,380,307 | 14,524,316 |
| Total sequences | 89,352 | 71,185 | 160,537 |
| Total ditags identified | 280,487 | 260,359 | 540,846 |
| Total unique ditags | 46,354 (100) | 35,536 (100) | 81,890 (100) |
| Mapped ditags | 40,985 (88.4) | 29,964 (84.3) | 70,949 (86.6) |
| Human genome | 40,380 (87.1) | 29,447 (82.9) | 69,827 (85.3) |
| sequences (HG18) | | | |
| Perfect match | 37,318 (80.5) | 26,564 (74.8) | 63,882 (78.0) |
| 1-base mismatch | 2,134 (4.6) | 1,850 (5.2) | 3,966 (4.8) |
| SNP | 166 (0.4) | 83 (0.2) | 249 (0.3) |
| Homopolymer | 772 (1.7) | 958 (2.7) | 1,730 (2.1) |
| Chimpanzee genome | 277 (0.6) | 181 (0.5) | 458 (0.6) |
| sequences | | | |
| Human genome | 318 (0.7) | 328 (0.9) | 664 (0.8) |
| variations* | | | |
| GM15510 fosmid | 25 | 30 | 55 |
| sequences | | | |
| Celera human genome | 167 | 147 | 314 |
| sequences | | | |
| Venter genome | 269 | 274 | 543 |
| sequences | | | |
| Watson genome | 28 | 34 | 62 |
| sequences | | | |
| Trouble mapped ditags | 5,248 (11.6) | 5,533 (15.7) | 10,781 (13.3) |
| Two single tags | 3,509 (7.6) | 4,549 (12.8) | 8,058 (9.8) |
| mapped | | | |
| Same chromosome | 1,073 (2.3) | 2,091 (5.9) | 3,164 (3.9) |
| Different | 2,436 (5.3) | 2,458 (6.9) | 4,894 (6.0) |
| chromosomes | | | |
| Only one single tag | 1,739 (3.8) | 984 (2.8) | 2,723 (3.3) |
| mapped | | | |
| Both single tags don't | 121 (0.3) | 39 (0.1) | 160 (0.2) |
| map | | | |

*The 664 ditags map to 1,007 loci across diffeent genomes.
The ditag mapped to more than one individual genome was counted only once.

The ditags mapped solely to the chimpanzee genome sequences account for 0.6% of the total ditags (Data not shown). These ditags likely represent the human DNA fragments missed in the human genome reference sequences. The high mapping rate indicates that, under the given resolution, most of the DNA fragments in the GM15510 genome detected by ditags have the same structure as their corresponding ones in HG18.

Detecting shorter DNA fragments implies the high resolution for analyzing genome structure. Computational analysis shows that the proportion of the fragments shorter than 6 kb is dominant among the total fragments generated by many high frequent 6-base restriction enzymes (Table 2). To verify this feature, we analyzed the size distribution of the virtual DNA fragments in HG18 that were detected by the experimental ditags. The results show that the majority of the fragments have shorter sizes (FIG. 15). Setting 6 kb as the cut-off, 93% of the detected DNA fragments are shorter than 6 kb, and 43% are shorter than 1 kb. These rates are even higher than those present in the HG18 in which 72% of the fragments are shorter than 6 kb and 23% of fragments are shorter than 1 kb. The increased rate of shorter DNA fragments is mostly due to the use of plasmid vector for the cloning that preferably clones the shorter fragments. Such size distribution ensures the kilobase resolution for analyzing genome structure.

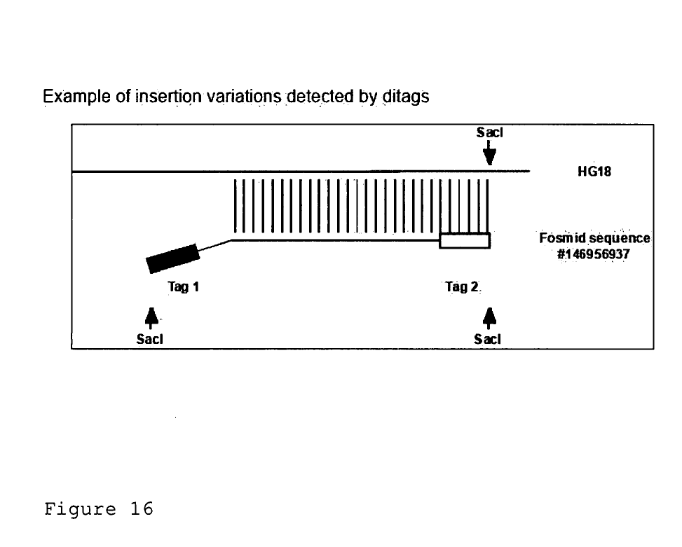

A total of 2,298,774 end sequences were generated from GM15510 fosmid library (International Human genome study consortium, 2004). The variations affecting smaller regions in GM15510 genome, if existing, could be present in the end sequences, and many could be detected by the ditags. We investigated this possibility. Reference ditags were extracted from the sequences containing at least two SacI or HindIII sites that are detectable by ditags. The experimental ditags that do not map to HG18 were mapped against these reference ditags. A total of 55 experimental ditags were identified to map to the fosmid end sequences. Comparing each mapped sequence to HG18 shows various variations including novel DNA sequence, deletion and insertion, and ditag sequence change including mutations in the restriction site that controls the release of the tags from the genomic DNA, and mismatches in the tag sequences (FIG. 16). The average length of the mapped 55 variation sequences is 289 bps. Although these variations were included in the original fosmid sequences, they were not identified as variations at the 40-kb resolution by the fosmid study (Tuzun et al. 2005) but detected by the ditag approach with its increased resolution. Comparing the ditag-detected 55 variations with the 297 variations (including the 40 fully sequenced fosmid clones) detected in the GM15510 by the fosmid study shows no overlapping. This is likely attributed to the limited genome coverage by the collected ditags, and the single-base resolution used for ditag mapping (See Table 5 below).

Recently, three sets of the human genome sequences become publicly available, including the assembled Celera human genome sequences, the unassembled Venter genome sequences that are at several kilobases per sequence, and the unassembled Watson genome sequences that are the raw 454 sequences of about 250 bp per sequence. These sequences provide a rich source to identify the experimental ditags originated from the variable regions in individual human genomes. We extracted reference ditags from these three sets of human genome sequences, and compared the experimental ditags that do not mapped to HG18 to these reference ditags. In total, 572 ditags mapped to the Celera genome sequences, 868 ditags to the Venter genome sequences, and 100 ditags to the Watson genome sequence (Table 4, Supplementary Table 5). The relatively higher mapping rate to the Venter genome sequences is likely due to the unassembled nature of the sequences that contributed more reference ditags than the assembled sequences; the lower mapping rate to the Watson genome sequences is due to the short length of the 454 sequences that many sequences don't contribute reference ditags since they don't have two (SacI or HindIII) restriction sites for reference ditag extraction.

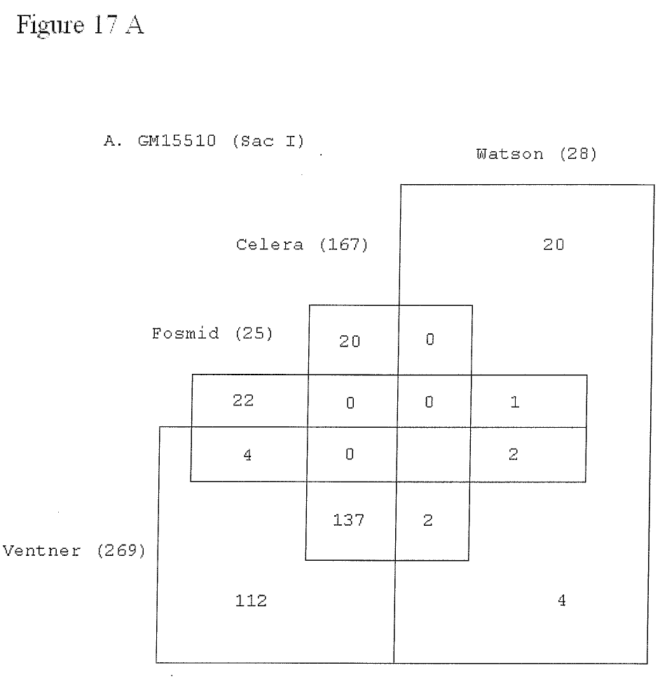

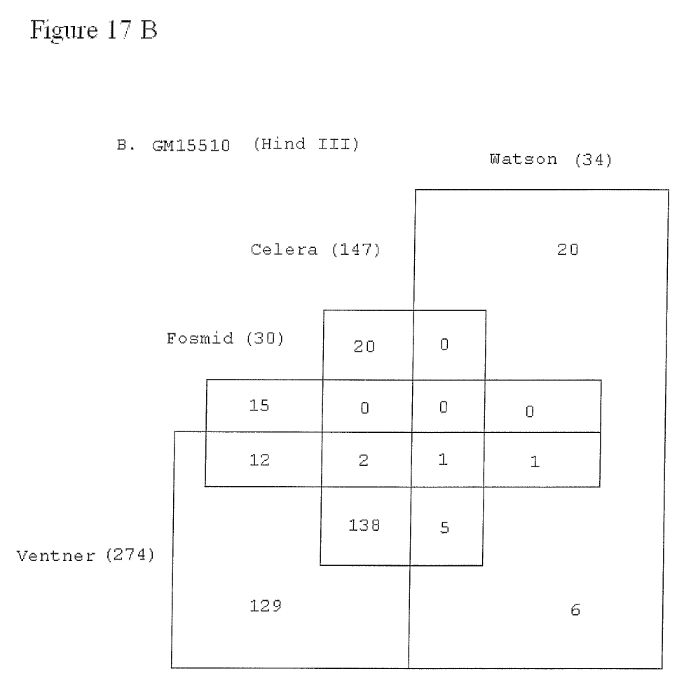

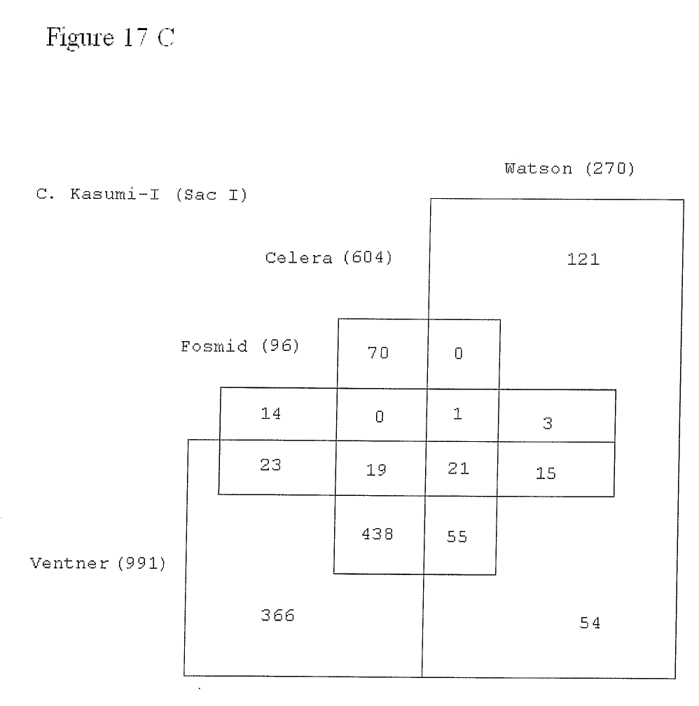

Overall, in the ditags not mapped to HG18 or chimpanzee genome sequence, 646 ditags mapped to 975 loci across the four individual genomes that contain the genome variations at kilobase levels (FIG. 17, A and B). By comparing the ditags mapped to the four genomes and the ditag mapped to the HG18, the variation rate is 0.8% (646/81,890). This rate is close to the 1% variation in GM15510 genome determined at the 40 kb resolution (Tuzun et al. 2005). Of these mapped ditags, most mapped to more than one individual genome. For example, of the 169 SacI ditags mapped to the Celera genome, 149 also mapped to the Venter genome, 10 to the Watson genome, 4 to the GM15510 genome, and 2 mapped to all four individual genomes. The ditags mapped to more than one individual genome represent the genome variations commonly existing in different individual genomes.

Cancer genome structure can be substantially alternated from the normal genome. We used Kasumi-1 cells as a model to test the power of DGS for detecting genome alternations in a cancer genome. Kasumi-1 is a leukemic cell line whose genome varies greatly from the normal genome, as reflected by its complicated karyotype (Asou et al. 1991; Horsley et al. 2006). We collected ditags from Kasumi-1 SacI DNA fragments by using a single 454 sequencing run that doubled the ditag detection over the GM15510 SacI restriction fragments (Table 6). The ditags collected provide 39% genome coverage when referring to the fragments <6 kb in HG18, or 28% when referring to the total genome fragments in HG18. The experimental ditags were processed by using the established ditag mapping procedure.

TABLE 6

| | |
|---|---|
| Mapping Location | Length |

| | | | |
|---|---|---|---|
| Ditag | Tag 1 | Tag 2 | (bp) |

| | | | |
|---|---|---|---|
| GAGCTCAGGGTGTGCC/TCCCTGGTTTGAGCTC | 11718065 | 11717838 | 227 |
| SEQ ID NO: 179, 180 | | | |
| GAGCTCCCCCTTCATGA/GCCCTAACGAGAGCTC | 3166629 | 31666392 | 237 |
| SEQ ID NO: 181, 182 | | | |
| GAGCTCCCCAGTATGT/TCAATTTTTGGAGCTC | 10541454 | 10544669 | 243 |
| SEQ ID NO: 5, 6 | | | |
| GAGCTCCCTCAATTTC/TTAGGCTTGTGAGCTC | 57414806 | 57414230 | 576 |
| SEQ ID NO: 183, 184 | | | |
| GAGCTCCTAGAATGTA/TCAGCCCTGTGAGCTC | 10575149 | 10577078 | 582 |
| SEQ ID NO: 7, 8 | | | |
| GAGCTCTCGTTAGGGC/TCATGAAGGGGAGCTC | 3166392 | 3166629 | 1407 |
| SEQ ID NO: 1, 2 | | | |
| GAGCTCACAGGGCTGA/TACATTCTAGGAGCTC | 10577078 | 10575149 | 1929 |
| SEQ ID NO: 185, 186 | | | |
| GAGCTCACTCTTGGAT/TGGATCACTTGAGCTC | 57426519 | 57431783 | 1935 |
| SEQ ID NO: 31, 32 | | | |
| GAGCTCTCATGTCTGG/TCTGCCTGCCGAGCTC | 57425118 | 57426519 | 3221 |
| SEQ ID NO: 187, 188 | | | |
| GAGCTCACAAGCCTAA/GAAATTGAGGGAGCTC | 57414230 | 57414806 | 5270 |
| SEQ ID NO: 27, 28 | | | |
| GAGCTCTCATGCCTTT/TTTGCTCCCGGAGCTC | 11924155 | 11929821 | 5666 |
| SEQ ID NO: 189, 190 | | | |

The results show the following features:

Large genome size. Under a defined scale of ditag sequencing, the ratio between the number of total ditag copies and the number of total unique ditags reflects the relative size of different genomes. The lower ratio represents the larger size and the higher ratio represents the smaller size of the genome. In Kasumi-1, the ratio is 2 to 1 (350,005 SacI ditag copies generate 168,281 unique ditags) whereas in GM15510 ditags, the ratio is 6 to 1 (280,409 SacI ditag copies generate 46,354 unique ditags). Consistent with the results from Kasumi-1 karyotyping which shows many extra genome contents over the standard ones, such as the trisomy 3 and 8, the size of the Kasumi-1 genome is substantially larger than the GM1510 genome.

High frequent genome structural alternation. This is reflected by the high rate of Kasumi1 ditags not mapped to the human genome reference sequences. Compared to the 86.6% in GM15510, only 73.7% are the mapped ditags in Kasumi-1 ditags. The difference is due largely to the lower rate of the perfectly mapped ditags: in contrast to 78% in GM15510, only 65.1% of Kasumi-1 ditags are the perfectly mapped ditags. The lower mapping rate leads to higher rate of trouble-mapped ditags: 26.3% of Kasumi-1 ditags are the trouble-mapped ditags, comparing to the 13.3% of the GM15510 ditags.

Presence of normal genome variations. Comparing the ditags to the four additional human genome sequences identified 1,198 ditags that represent the variations in normal human genomes (FIG. 17, C). The rate (0.7%) is similar to the one observed in GM15510 ditag mapped variations (0.8%). Considering that the scale of Kasumi-1 SacI ditag collection doubled that of GM15510, we tested if the increased ditag detection could detect the variations in GM15510 genome identified by fosmid sequencing. We compared the ditags with the reference ditags extracted from the 33 fully sequenced fosmid clones of the 297 variations. Of the 307 SacI reference ditags from these clones, 123 are mapped by the Kasumi-1 ditags, of which 116 ditags are common to the HG18 whereas 7 ditags are located in the variations not present in HG18 but in 5 fosmid clones including 4 insertions and 1 deletion (See Table 7 below).

TABLE 7
Kasumi-1 ditags detect GM15510 variations,
revealed by fosmid sequences
A. Summary of the mapping results

| Items | Number |
|---|---|
| Fully sequenced fosmid clones* | 33 |
| Reference ditags from the sequences | 307 |
| Reference ditags mapped by Kasumi-1 diag | 123 |
| Mapped reference ditags common to HG18 | 116 |
| Mapped reference ditags only in fosmid sequences | 7 |
| Detected variations | 4 |
| Type | Insertion |
| Position of mapped ditags | |
| Inside the insertion | 3 |
| Across the junction | 4 |

*Of the 40 fully sequenced clones, only 33 have at least 2 SacI sites for releasing reference ditags.

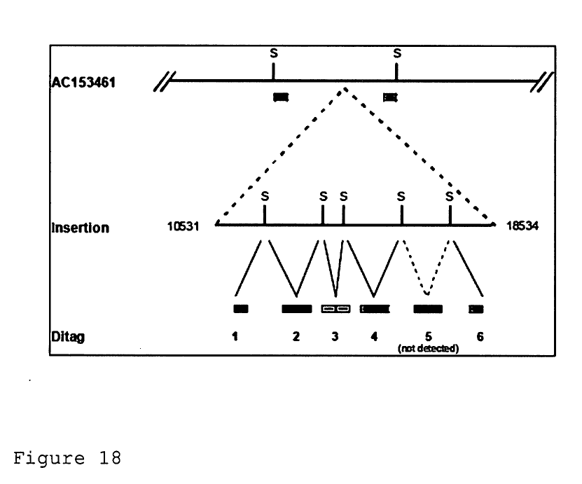

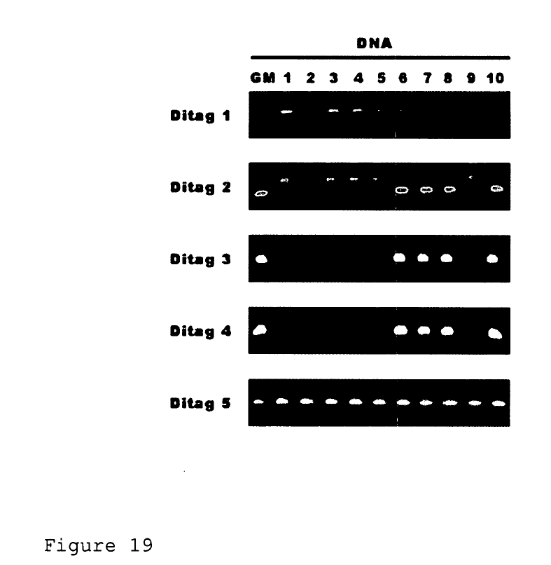

Taking the fosmid variation AC153461 as the example, this variation maps to chromosome 7 but contain an 8,002 bp insertion that does not map to HG18. Of the 10 reference ditags extracted from this sequence, 4 are shared with HG18 representing normal sequences but 6 only in AC153461 representing the insertion. Of these 6 reference ditags, 5 were detected by Kasumi-1 ditags, of which 2 across the junctions between the normal sequences and the insertion and 3 are purely from the insertion (FIG. 18). The mapping of Kasumi-ditags to the normal variation ditags indicates that the Kasumi-1 genome contains the genome variations present in the normal individual genomes.

The inventors found remaining chromosome Y fragments in the Kasumi-1 genome. Kasumi-1 cells originated from a male, but karyotype analyses consistently show that the whole Y chromosome is lost from the cell (Asou et al. 1991). However, 11 ditags map specifically to the reference ditags of chromosome Y (Data not shown). The presence of ditags from chromosome Y indicates that these chromosome Y fragments did not disappear, but integrated into other chromosome(s) in the Kasumi-1 genome.

The foregoing descriptions and examples should be considered as illustrative only of the principles of the invention. Since numerous applications of the present invention will readily occur to those skilled in the art, it is not desired to limit the invention to the specific examples disclosed or the exact construction and operation shown and described. Rather, all suitable modifications and equivalents may be resorted to, falling within the scope of the invention.

Having described the invention, many modifications thereto will become apparent to those skilled in the art to which it pertains without deviation from the spirit of the invention as defined by the scope of the appended claims. The disclosures of U.S. patents, patent applications, and all other references cited above are all hereby incorporated by reference into this specification as if fully set forth in its entirety.

### REFERENCES

1. The Genome Sequencing Consortium. Initial sequencing and analysis of the human genome. Nature 409, 860-921 (2001).
2. Venter, J. C. et al. The sequence of the human genome. Science. 291, 1304-1351 (2001).
3. Sachidanandam, R. et al. International SNP Map Working Group. A map of human genome sequence variation containing 1.42 million single nucleotide polymorphisms. Nature 409, 928-933 (2001).
4. Sebat, J. et al. Large-scale copy number polymorphism in the human genome. Science 305, 525-528 (2004).
5. Iafrate, A. J. et al. Detection of large-scale variation in the human genome. Nat. Genet. 36, 949-951 (2004).
6. Tuzun, E. et al. Fine-scale structural variation of the human genome. Nat Genet. 37, 727-732 (2005).
7. Feuk, L., Carson, A. R., & Scherer, S. W. Structural variation in the human genome. Nat Rev Genet. 7, 85-97 (2006).
8. McCarroll, S. A. et al. International HapMap Consortium. Common deletion polymorphisms in the human genome. Nat Genet. 38, 86-92 (2006).
9. Eichler, E. E. Widening the spectrum of human genetic variation. Nat Genet. 38, 9-11, (2006).
10. Pinkel, D. et al. High resolution analysis of DNA copy number variation using comparative genomic hybridization to microarrays. Nat Genet. 20, 207-211 (1998).
11. Bertone, P. et al. Global identification of human transcribed sequences with genome tiling arrays. Science 306, 2242-2246 (2004).
12. Cheng, J. et al. Transcriptional maps of 10 human chromosomes at 5-nucleotide resolution. Science 308, 1149-1154 (2005).
13. Kim, T. H. et al. A high-resolution map of active promoters in the human genome. Nature 436, 876-880 (2005).
14. Shendure J, et al. Accurate multiplex polony sequencing of an evolved bacterial genome. Science 309, 1728-1732 (2005).
15. Anantharaman, T. S., Mysore, V., & Mishra, B. Fast and cheap genome wide haplotype construction via optical mapping. Pac Symp Biocomput. 385-396 (2005).
16. Margulies, M. et al. Genome sequencing in microfabricated high-density picolitre reactors. Nature 437, 376-380 (2005).
17. Asou, H. et al. Establishment of a human acute myeloid leukemia cell line (Kasumi-1) with 8;21 chromosome translocation. Blood 77, 2031-2036 (1991).
18. Wang, T. L. et al. Digital karyotyping. Proc Natl Acad Sci USA. 99, 16156-16161, (2002).
19. Wei, C. L. et al. A global map of p53 transcription-factor binding sites in the human genome. Cell 124, 207-219 (2006).
20. Zhang Y, et al. Genomic DNA breakpoints in AML1/RUNX1 and ETO cluster with topoisomerase II DNA cleavage and DNase I hypersensitive sites in t(8;21) leukemia. Proc Natl Acad Sci USA. 99, 3070-3075 (2002).

1. A system for collecting genetic information using DNA sequences comprising the steps of:
1) collecting two short tags from both ends of DNA fragments to form a ditag;
2) using the 454 sequencing system for maximal collection of ditags at the genome scale;
3) identifying the DNA fragments in the human genome sequences that originated the ditags and identify the DNA fragments that are different from those in the reference human genome;
4) confirming the mapping results by using the ditag sequences directly as the sense and antisense primers in a PCR expansion to detect the original DNA fragments; and
5) performing computational and experimental analysis of DGS results.

2. A method for determining of the genome origin of a Ditag through the Ditagmap reference database comprising the steps of:
a. dividing the identified ditags into three groups, and classifying as mapped ditags, those ditags having been identified with reference ditags in a one to one correspondence, and with mismatches up to two bases, of which the p values are higher than the cutoff of 1.0e⁻⁵; classifying as trouble-mapped ditags, those identified ditags of which the combined p values of mapping two single tags in reference ditag database are higher than the cutoff of 1.0e⁻³, or, any single tag mapping p value is larger than 1.0e⁻³, which allows at most one mismatch with reference tags; and classifying as unmapped ditags, those ditags having p values that are less than the cutoff 1.0e⁻³ when their two single tags are mapped to reference ditag database;
b. selecting a reference ditag having a 32-bp tag from the 5′ end and a 32-bp tag from the 3′ end of a virtual DNA fragment;
c. searching the DitagMap reference database for the experimental ditag, by comparing experimental ditags having a sequence shorter than 32 bp with reference ditags of the same length, and counting total mismatches without allowing gaps, wherein ditags having a sequence longer than 31 bp are compared with reference ditags with extra bases, such that the 16-bp in both ends of the longer ditags are aligned with the ends of each reference ditag, then the extra bases between the two 16-bp are compared with the bases in the reference ditag, and those bases with matches are assigned to the corresponding single tag;
d. identifying length of experimental ditag and the mismatches with each reference ditag;
e. calculating the probability of an experimental ditag/tag (w_{ob}) mapping in the reference database is calculated by using the formula: p-score=
$$1 - {\prod }_{{w}_{i} ∈ R} ( 1 - p ( {w}_{ob} , {w}_{i} ) ) , \\ while$$
$$p ( {w}_{ob} , {w}_{i} ) = {p}_{err}^{m} * {( 1 - {p}_{err} )}^{( L - m )} ,$$
where R represents the whole set of ditags/tags obtained from the reference human genome, wᵢ represents the ith ditag/tag in it, L is the length of the experimental ditag or single tag, with length of perfect match or one-base mismatch, m is the number of the mismatched base(s) between w_{ob} and wᵢ. pₑᵣᵣ is the rate of sequencing error and/or SNPs, and
f) identifying a ditag as mapped in the reference.

3. The method of claim 2, wherein the identifying the genomic origin of at least one ditag represents a genomic variation between individuals.

4. A method for producing and collecting ditag sequence information comprising the following steps:
a) obtaining a genomic DNA sample;
b) fragmenting the genomic DNA sample by restriction enzyme digestion;
c) cloning the DNA fragments generated in step b) into plasmid vectors to generate a genomic DNA library;
d) digesting the library using the restriction enzyme MmeI such that two short tags are retained on each site of the cloned DNA fragment in the same plasmid vector in a tag-vector-tag orientation;
e) religating the tag-vector tag fragments to form a ditag;
f) releasing the ditags formed in step e) from the vectors by digestion with a restriction enzyme;
g) concatemerizing the individual ditags having a suitable length for sequencing;
h) sequencing the concatemerized ditags using a 454 sequencing system;
i) extracting the ditags from the sequences based on the identification of their restriction sites;
j) mapping the ditags extracted from step i) to a reference ditag database where restriction fragments of known reference genome sequences are stored;
k) determining whether the ditag has a counterpart in the reference ditag database and identifying those ditags which have counterpart sequences to mapped; and
l) identifying the ditags which do not have a counterpart in the reference ditag database as trouble-mapped ditags.


SEQUENCE LISTING (204 sequences)

SEQ ID NO: 1 - DNA, 16 bp, Homo sapiens
gagctctcgttagggc

SEQ ID NO: 2 - DNA, 16 bp, Homo sapiens
tcatgaaggggagctc

SEQ ID NO: 3 - DNA, 16 bp, Homo sapiens
gagctcaggagattga

SEQ ID NO: 4 - DNA, 16 bp, Homo sapiens
aggatcacttgagctc

SEQ ID NO: 5 - DNA, 16 bp, Homo sapiens
gagctccccagtatgt

SEQ ID NO: 6 - DNA, 16 bp, Homo sapiens
tcaatttttggagctc

SEQ ID NO: 7 - DNA, 16 bp, Homo sapiens
gagctcctagaatgta

SEQ ID NO: 8 - DNA, 16 bp, Homo sapiens
tcagccctgtgagctc

SEQ ID NO: 9 - DNA, 16 bp, Homo sapiens
gagctctgtcccagga

SEQ ID NO: 10 - DNA, 16 bp, Homo sapiens
ctccctctgggagctc

SEQ ID NO: 11 - DNA, 16 bp, Homo sapiens
gagctcttggccaatg

SEQ ID NO: 12 - DNA, 16 bp, Homo sapiens
atgtaagtatgagctc

SEQ ID NO: 13 - DNA, 16 bp, Homo sapiens
gagctcagactgaggg

SEQ ID NO: 14 - DNA, 16 bp, Homo sapiens
cttggacagtgagctc

SEQ ID NO: 15 - DNA, 16 bp, Homo sapiens
gagctcagaaaatttg

SEQ ID NO: 16 - DNA, 16 bp, Homo sapiens
gaatggaggggagctc

SEQ ID NO: 17 - DNA, 16 bp, Homo sapiens
gagctcaagaccagcc

SEQ ID NO: 18 - DNA, 16 bp, Homo sapiens
tctccaatgagagctc

SEQ ID NO: 19 - DNA, 16 bp, Homo sapiens
gagctctgtccacatg

SEQ ID NO: 20 - DNA, 16 bp, Homo sapiens
gggagctgaggagctc

SEQ ID NO: 21 - DNA, 16 bp, Homo sapiens
gagctctctgttgccc

SEQ ID NO: 22 - DNA, 16 bp, Homo sapiens
acattaatctgagctc

SEQ ID NO: 23 - DNA, 16 bp, Homo sapiens
gagctccctcagccct

SEQ ID NO: 24 - DNA, 16 bp, Homo sapiens
cttctcaaatgagctc

SEQ ID NO: 25 - DNA, 16 bp, Homo sapiens
gagctctgaagatata

SEQ ID NO: 26 - DNA, 16 bp, Homo sapiens
tcaacctcctgagctc

SEQ ID NO: 27 - DNA, 16 bp, Homo sapiens
gagctcacaagcctaa

SEQ ID NO: 28 - DNA, 16 bp, Homo sapiens
gaaattgagggagctc

SEQ ID NO: 29 - DNA, 16 bp, Homo sapiens
gagctctcatgtctgg

SEQ ID NO: 30 - DNA, 16 bp, Homo sapiens
tctgcctgccgagctc

SEQ ID NO: 31 - DNA, 16 bp, Homo sapiens
gagctcactcttggat

SEQ ID NO: 32 - DNA, 16 bp, Homo sapiens
tggatcacttgagctc

SEQ ID NO: 33 - DNA, 16 bp, Homo sapiens
gagctctgggcgcgtg

SEQ ID NO: 34 - DNA, 16 bp, Homo sapiens
cctctactgagagctc

SEQ ID NO: 35 - DNA, 16 bp, Homo sapiens
gagctcaggcgggtcc

SEQ ID NO: 36 - DNA, 16 bp, Homo sapiens
ccctcctcatgagctc

SEQ ID NO: 37 - DNA, 16 bp, Homo sapiens
gagctctgcggggcgc

SEQ ID NO: 38 - DNA, 16 bp, Homo sapiens
tgggtgaaccgagctc

SEQ ID NO: 39 - DNA, 16 bp, Homo sapiens
gagctcactgcttgct

SEQ ID NO: 40 - DNA, 16 bp, Homo sapiens
gagctaggctgagctc

SEQ ID NO: 41 - DNA, 16 bp, Homo sapiens
gagctctcttctgcac

SEQ ID NO: 42 - DNA, 16 bp, Homo sapiens
gagaagttaggagctc

SEQ ID NO: 43 - DNA, 16 bp, Homo sapiens
gagctcagggtatcta

SEQ ID NO: 44 - DNA, 16 bp, Homo sapiens
tctgagtcatgagctc

SEQ ID NO: 45 - DNA, 16 bp, Homo sapiens
gagctcatttctccct

SEQ ID NO: 46 - DNA, 16 bp, Homo sapiens
gagcacgtgggagctc

SEQ ID NO: 47 - DNA, 16 bp, Homo sapiens
gagctcccacgtgctc

SEQ ID NO: 48 - DNA, 16 bp, Homo sapiens
agggagaaatgagctc

SEQ ID NO: 49 - DNA, 16 bp, Homo sapiens
gagctcgcccctgagc

SEQ ID NO: 50 - DNA, 16 bp, Homo sapiens
cccagtgggtgagctc

SEQ ID NO: 51 - DNA, 16 bp, Homo sapiens
gagctcagggcccagg

SEQ ID NO: 52 - DNA, 16 bp, Homo sapiens
gggtcaaggtgagctc

SEQ ID NO: 53 - DNA, 16 bp, Homo sapiens
gagctcatgaggaggg

SEQ ID NO: 54 - DNA, 16 bp, Homo sapiens
ggacccgcctgagctc

SEQ ID NO: 55 - DNA, 16 bp, Homo sapiens
gagctctgcaaacctt

SEQ ID NO: 56 - DNA, 16 bp, Homo sapiens
taaggacagagagctc

SEQ ID NO: 57 - DNA, 16 bp, Homo sapiens
gagctcagcagcaggt

SEQ ID NO: 58 - DNA, 16 bp, Homo sapiens
gtgatggctggagctc

SEQ ID NO: 59 - DNA, 16 bp, Homo sapiens
gagctcactctagtca

SEQ ID NO: 60 - DNA, 16 bp, Homo sapiens
ccaggaatcagagctc

SEQ ID NO: 61 - DNA, 16 bp, Homo sapiens
gagctcgaaccaggga

SEQ ID NO: 62 - DNA, 16 bp, Homo sapiens
ggcacaccctgagctc

SEQ ID NO: 63 - DNA, 16 bp, Homo sapiens
gagctcgaaccaggga

SEQ ID NO: 64 - DNA, 16 bp, Homo sapiens
ggcacaccctgagctc

SEQ ID NO: 65 - DNA, 16 bp, Homo sapiens
gagctccggacttagc

SEQ ID NO: 66 - DNA, 16 bp, Homo sapiens
accttccagggagctc

SEQ ID NO: 67 - DNA, 16 bp, Homo sapiens
gagctcagggtgtgcc

SEQ ID NO: 68 - DNA, 16 bp, Homo sapiens
tccctggttcgagctc

SEQ ID NO: 69 - DNA, 16 bp, Homo sapiens
gagctcttcgaccacc

SEQ ID NO: 70 - DNA, 16 bp, Homo sapiens
tcgaactcctgagctc

SEQ ID NO: 71 - DNA, 16 bp, Homo sapiens
gagctcatagaggcag

SEQ ID NO: 72 - DNA, 16 bp, Homo sapiens
tcgaactcctgagctc

SEQ ID NO: 73 - DNA, 16 bp, Homo sapiens
gagctcgcggtgcctt

SEQ ID NO: 74 - DNA, 16 bp, Homo sapiens
caccagtccagagctc

SEQ ID NO: 75 - DNA, 16 bp, Homo sapiens
gagctccgtcaggtca

SEQ ID NO: 76 - DNA, 16 bp, Homo sapiens
ctgaggtcaggagctc

SEQ ID NO: 77 - DNA, 16 bp, Homo sapiens
gagctcttactgtgct

SEQ ID NO: 78 - DNA, 16 bp, Homo sapiens
atcctgcacagagctc

SEQ ID NO: 79 - DNA, 16 bp, Homo sapiens
gagctcaggaggttga

SEQ ID NO: 80 - DNA, 16 bp, Homo sapiens
gaaggagcaggagctc

SEQ ID NO: 81 - DNA, 16 bp, Homo sapiens
gagctcatatgtatcc

SEQ ID NO: 82 - DNA, 16 bp, Homo sapiens
aggatcacttgagctc

SEQ ID NO: 83 - DNA, 16 bp, Homo sapiens
gagctcacacgatgga

SEQ ID NO: 84 - DNA, 16 bp, Homo sapiens
tcagagctgagagctc

SEQ ID NO: 85 - DNA, 16 bp, Homo sapiens
gagctcgcataccaca

SEQ ID NO: 86 - DNA, 16 bp, Homo sapiens
tgtgagggttgagctc

SEQ ID NO: 87 - DNA, 16 bp, Homo sapiens
gagctcgtgtctcctc

SEQ ID NO: 88 - DNA, 16 bp, Homo sapiens
gtctctggatgagctc

SEQ ID NO: 89 - DNA, 16 bp, Homo sapiens
gagctcaagtagtcct

SEQ ID NO: 90 - DNA, 16 bp, Homo sapiens
ggtgtgctatgagctc

SEQ ID NO: 91 - DNA, 16 bp, Homo sapiens
gagctcaccacagctc

SEQ ID NO: 92 - DNA, 16 bp, Homo sapiens
gctgagcgtggagctc

SEQ ID NO: 93 - DNA, 16 bp, Homo sapiens
gagctcttcaggtcag

SEQ ID NO: 94 - DNA, 16 bp, Homo sapiens
aacaccgtcagagctc

SEQ ID NO: 95 - DNA, 16 bp, Homo sapiens
gagctcagggaggaca

SEQ ID NO: 96 - DNA, 16 bp, Homo sapiens
tgccgcccgcgagctc

SEQ ID NO: 97 - DNA, 16 bp, Homo sapiens
gagctctgactatgga

SEQ ID NO: 98 - DNA, 16 bp, Homo sapiens
ccatactggggagctc

SEQ ID NO: 99 - DNA, 16 bp, Homo sapiens
gagctccgtatcacgc

SEQ ID NO: 100 - DNA, 16 bp, Homo sapiens
aggctgaggtgagctc

SEQ ID NO: 101 - DNA, 16 bp, Homo sapiens
gagctcctgaccttgt

SEQ ID NO: 102 - DNA, 16 bp, Homo sapiens
atgtgtgccagagctc

SEQ ID NO: 103 - DNA, 16 bp, Homo sapiens
gagctctgctagatga

SEQ ID NO: 104 - DNA, 16 bp, Homo sapiens
cagatcgcttgagctc

SEQ ID NO: 105 - DNA, 16 bp, Homo sapiens
gagctctactgttgct

SEQ ID NO: 106 - DNA, 16 bp, Homo sapiens
ccagacctaggagctc

SEQ ID NO: 107 - DNA, 16 bp, Homo sapiens
gagctccagcagccac

SEQ ID NO: 108 - DNA, 16 bp, Homo sapiens
tgaccctggggagctc

SEQ ID NO: 109 - DNA, 16 bp, Homo sapiens
gagctctactgttgct

SEQ ID NO: 110 - DNA, 16 bp, Homo sapiens
ccagacctaggagctc

SEQ ID NO: 111 - DNA, 16 bp, Homo sapiens
gagctctctccgtgcg

SEQ ID NO: 112 - DNA, 16 bp, Homo sapiens
gaaaccacaggagctc

SEQ ID NO: 113 - DNA, 16 bp, Homo sapiens
gagctcgagctcaggc

SEQ ID NO: 114 - DNA, 16 bp, Homo sapiens
agtgtagttcgagctc

SEQ ID NO: 115 - DNA, 16 bp, Homo sapiens
gagctcgcgggcggca

SEQ ID NO: 116 - DNA, 16 bp, Homo sapiens
gctaagtccggagctc

SEQ ID NO: 117 - DNA, 16 bp, Homo sapiens
gagctcgcgggcggca

SEQ ID NO: 118 - DNA, 16 bp, Homo sapiens
tgtcctccctgagctc

SEQ ID NO: 119 - DNA, 16 bp, Homo sapiens
gagctcgcgggcggca

SEQ ID NO: 120 - DNA, 16 bp, Homo sapiens
gctaagtccggagctc

SEQ ID NO: 121 - DNA, 109 bp, Homo sapiens
gagctctgggcgcgtgccaaggaagagtgcaccctaggctgggagggtggtgcacaccgcgatcctcaggctttctgggaccaaccctgccgacctctactgagagctc

SEQ ID NO: 122 - DNA, 313 bp, Homo sapiens
gagctcaggcgggtccagatgaccctgtcctccttttgtaacagccagagtccaggatgctttgcccagggcattgggctggcactgcagaggcctgggggatggggcgacacctgggacatggctggtgggaattgttctaggaaacctcagggattctccctggacctgtcaaagccccttccctgtttcttctgaggctgtgtcccccccactcgcacaagggtcctttctatgcctgctcccctgataaatgtcatctgcctgctctagaatggcttccagaccccacagaccccctcctcatgagctc

SEQ ID NO: 123 - DNA, 112 bp, Homo sapiens
gagctctgcggggcgctgttggcggtgggtgaaccgagcctccgcggggtgctgttggcggtgggtgaaccgagttctgcggggcgctgttggcggtgggtgaaccgagctc

SEQ ID NO: 124 - DNA, 189 bp, Homo sapiens
gagctcactgcttgctgaggcaagagcgcctcctcatggctgtcaggcaacagggcgcttgggtgctgctggggaatccagccgggccagggtgagaggccagcgccccaagatgtggaggccctgagaccttggcatcagcccaagaacctcctcctaggggctggcagtgggagctaggctgagctc

SEQ ID NO: 125 - DNA, 59 bp, Homo sapiens
gagctctcttctgcacccgcattggagggtggagattgttggggagaagttaggagctc

SEQ ID NO: 126 - DNA, 57 bp, Homo sapiens
gagctcagggtatctattcatttatttagggtactgggtcctctgagtcatgagctc

SEQ ID NO: 127 - DNA, 82 bp, Homo sapiens
gagctcatttctccctgggtgtgtgtgtgtgtgtgtgtgtgtgtgtgagtgaaagacagagacacagagcacgtgggagctc

SEQ ID NO: 128 - DNA, 70 bp, Homo sapiens
gagctcatttctccctgggtgtgtgtgtgtgtgtgtgagaaagacagagacacagagcacgtgggagctc

SEQ ID NO: 129 - DNA, 130 bp, Homo sapiens
gagctcatttctccctgggtgtgtgtgtgtgtgtgacacacagagcacgtgggagcccatttctccctgggtgtgtgtgtgtgtgtgtgtgtgtgtgtgtgtgtgtgacacacagagcacgtgggagctc

SEQ ID NO: 130 - DNA, 78 bp, Homo sapiens
gagctcccacgtgctctgtctctgtcttacgcacacacacacacacacacacacacacccccagggagaaatgagctc

SEQ ID NO: 131 - DNA, 70 bp, Homo sapiens
gagctcccacgtgctctgcgtctctgtctttcacacacacacacacacacacccagggagaaatgagctc

SEQ ID NO: 132 - DNA, 232 bp, Homo sapiens
gagctcgcccctgagcctgaggagacctgggtggcggagacgctgtgtggcctcaagatgaggcgagcgacggcgagtgtcgctcgtgctcctgagtactacgaggccttcaacaggctgcttggtaggaggaccaccccagagagcacctccaatcctgttctttctaaagaggaaacttccaataaccacacttttccaatgggaaaaatatgccccagtgggtgagctc

SEQ ID NO: 133 - DNA, 74 bp, Homo sapiens
gagctcagggcccagggctgggggccccccagggccagccctcttggatcttcggatggggtcaaggtgagctc

SEQ ID NO: 134 - DNA, 314 bp, Homo sapiens
gagctcatgaggagggggtctgtggggtctggaagccattctagagcaggcagatgacatttatcaggggagcaggcatagaaaggatccttgtgtgagtggggggacacacagcctcagaagaaacagggaaggggctttgacaggtccagggagaatccctgaggtttcctagaacaattcccaccagccatgtcccaggtgtcaccccatcccccaggcctctgcagtgccagcccaatgccctgggcaaagcatcctggactctggctgttacaaaaggaggacagggtcatctggacccgcctgagctc

SEQ ID NO: 135 - DNA, 682 bp, Homo sapiens
gagctctgcaaaccttattctcggcaaatgacataattggcggacgaaaaaattttttacaaaacatcatttaatttctggaattgtcccaaggcatacagcaatggagaacacctattcaggagatctgaataatctcaaaaagaacggttaacgtactatgggcttctggaggacccaccttattctttcaaccaaaccccttgtgtgatgagacctgtattctgggcaggtgggagtcagaagatgaagctccttgtcccccttactttctgatcttagaattataaattattcgctaggaggaggcaagacaccaacatttctcattcccccacctcagctctgtgttgaagaatctctaattctggcaaatgtggctgagaactcttgggatctcttcttccacccaaccccattcatagggcagaagctctacacaggtacagcagggtgaaaattctgggcccaactgtcctcaaaccagcttgcttataggatggagattcaatgccaggatagggaagccaagaagaccagagtctactgactatgctcatcaccccaagggtggagcagggtgttactccaagagaagcaagctaccatcctgacccccagcttcagagcagtggcacagagcttctcagagggagagaaaatcagtaaggacagagagctc

SEQ ID NO: 136 - DNA, 827 bp, Homo sapiens
gagctcagcagcaggttcaaaactcagttctcgcactgctaactgtgggagcaactaacaatctctctctgcctctgtttccccatctgcaaaatggatcagggtaggtcaactactgcagcaaacaaagcctggagctaaataccatccagctatgggccccacctcaatgagccacacagagtgctcagaacagcgcctagcacaaatctgtgcccactgcaggttcgcgaggatgggaaggctgtgccatccacaagagatagccagcggctatctcttcctccttgtcactgacaacatgaacgcagagcttgcagactagatggttgactgggcaaagaggggggagacagccttacagaagatggcagacacacggtgtgtgaggccggaagaaacactctgccctgagactcctctcctctatttggaactgcgcctggaggattgtattatatttccatgaattacatgccctccttacatccccaaacacaatggcattaacttccagatttcttttattgtggtgccaagagatgagcctgcattcactctctgtctcgcttgctgccacagggcaagggcagggctggggggcctcccaacctccatctcccctgcatcgtaccagctatgctagcactccatcagacagccccccacgagccacacgagctatgcgggggccatgtgactagaatctggccaataaagaggaattaacacagtgactggagctctgaaaagtcggcttcaatgaatcagctcccccaccccagccctctccattgcctagaatgaagaagtgatggctggagctc

SEQ ID NO: 137 - DNA, 295 bp, Homo sapiens
gagctcactctagtcattaagaatctcagtctcaatgtttgatttgtaagaaggcctcttgctccttgccaggtgcttcatcagtccactcttagatacaaaaaaaagatcctgctgtttctttatggtttccactgcccttttctcttaaacatcatactaaagtcaggcacatcttagaaatgcaactcatatttcatggttttctgattactaactgggaactaaatttgtagtccagggacaggactttgaagggagtaagtatcaaatatggggccaggaatcagagctc

SEQ ID NO: 138 - DNA, 233 bp, Homo sapiens
gagctcgaaccagggactctaggttccacggggcccagtgcaggggctgatgggaaggcactttcatccgtggggtacccaggccacacctctccgcggcggggttttttttttctttttctgtgacaggtgcctcacctctccttcctcaaaactcaccttcccctcacgggctttgtgtccccaaagtcccccttggggtgcacttggcggccgaggcacaccctgagctc

SEQ ID NO: 139 - DNA, 235 bp, Homo sapiens
gagctcgaaccagggaccccaggttccccggtgcccagcacaggggctgatgggaaggcactttcatccgtgggggacccaggccccacttctccacggcacgggttttttctttctttttctgtgacaggtgcctcacctctcctccctcaaaactcaccttcccctcatgggctttgtgcgcccaaaggccccccttggggtgcacttagcggctgaggcacaccctgagctc

SEQ ID NO: 140 - DNA, 200 bp, Homo sapiens
gagctcgaaccagggatgccagggtacccggggcgcagcgcaagggctgatggggagacactttcttccgtgggggacccaggccccgcttctttgaggcgcgttttttttttcctctgccccaggtgggtcaccttcccctcatgggccttctgcccgctttggggtacccctagcggccccaggcacaccctgagctc

SEQ ID NO: 141 - DNA, 64 bp, Homo sapiens
gagctccggacttagcggagctcgtacaccttccagggagctactgataccttccagggagctc

SEQ ID NO: 142 - DNA, 403 bp, Homo sapiens
gagctcagggtgtgcctcaggctctttaggaagtaacccaacttggacagaaggcccatgagttcctgttgaagtttgtgggaggagaggtgaggcagcaggggcagaaaaaaaaaaagaggaccgcgtatcagagaagcgggacctgggttccccacggatgaaagtgctttccattaggccctatgctgggcctggtggacactgggaccctgggttcaagccccgggtgcgcctcaggacagctgggataaccacaaacgaacaaaagtcatgaggggatgtggaggctctgagcatatatactatgctcatcgaatagcagtgcttgggcctcccggatgaagtgccatccatcgcccttccctgggctggacctgcttcttgttgaactgggtagctc

SEQ ID NO: 143 - DNA, 146 bp, Homo sapiens
gagctcttcgaccacctcggcctcccaaagtgctgggattgcaggagtgagccagcacgcccggctaattttcttatatatatatttgtagatatggtgggggggtcttgccatgttgcccagaatggtctcgaactcctgagctc

SEQ ID NO: 144 - DNA, 112 bp, Homo sapiens
gagctcatagaggcagacagacagacagagagagagaaggtctcactgggtcacccaggctggagtgcagtggtgcaatcacagctcactaaagcctcgaactcctgagctc

SEQ ID NO: 145 - DNA, 157 bp, Homo sapiens
gagctcatagaggcagtaaatcacagggtttgtgaagaggtcagcctgcctgggttttcattttttttttttttttaaaaacacagcctcactctgtcacccaggctggagtgaaggggcccaatcatagctcactgcagctcgaactcctgagctc

SEQ ID NO: 146 - DNA, 98 bp, Homo sapiens
gagctcgcggtgccttgatttccttgactgtaaattgggggacaataaagtactacctctagcactgatatttccttgcatgcaccagtccagagctc

SEQ ID NO: 147 - DNA, 170 bp, Homo sapiens
gagctccgtcaggtcaggagagaaaagcagggaggaatcctggctcattcattccacgaatatttcattcatttatttagtcagtcaggccgggtactgtggctcacacctgtaatcccaacactttgggaggctgagggggggggtggatcacctgaggtcaggagctc

SEQ ID NO: 148 - DNA, 175 bp, Homo sapiens
gagctccgtcaggtcaaggccgcagtgagccatgattgcaccactgcactccagcctcggtgatagagtgagaccctgtctcaaaaacaaaacaaggccaggtgcagtggctcatgcctataatcccagcactttgggaggccgagatgggtggatcacctgaggtcaggagctc

SEQ ID NO: 149 - DNA, 619 bp, Homo sapiens
gagctccgtcaggtcaccagtcattgcttggtgatgggaacagaagtgtggggatttggtttcccccagcctccacctcagtctgaattctccaaaacactctatgctgtttcttgccttcaagctttgggctctggctcttcgctcttcaaaaagggctttctcccaccccaggccctcctaacttcatccttcacagtacagcgttcgcagtcccaactggagtgaataagcttcctcccttctgcttccacacagctgggagggctgcacgcctcccctttgggttgtagtttctattcacatgcttatctgtccttctcacacaatgagctggggggctctcgggagctgggaccatatcttgttcatcttggtggcctccacagtgtccagcacagcataagtcaacaacacctgctaagaaaggaggataggaaaggtggaaagaagagaggctcctgtcacttcttctagagctgagttgtccagtatggtagccaccagccacatgtggctatttgaatttaaactaattgggccaggtgcagtggctcacgtctgtaatcccagcattttggaaggccaaggcgggtgcatcacctgaggtcaggagctc

SEQ ID NO: 150 - DNA, 51 bp, Homo sapiens
gagctcttactgtgcttatattgctcaaaatgcacatcctgcacagagctc

SEQ ID NO: 151 - DNA, 438 bp, Homo sapiens
  modified_base (109)..(141): a, c, g, t, unknown or other
gagctcaggaggttgaggctgcgagaaagagggtcatgcccactgaaatccagcccaggtgacacagtaagaccttgtcttcccccaccccccccaaaaaaaaaaaaannnnnnnnnnnnnnnnnnnnnnnnnnnnnnnnnttaggcattatgaaaaataaagaatacacaatattcgtgataaaagacttatatataaaaataaattatctttgaagatctatagacattaaatgcaatcctaaactcctacagcaaataaaaaattattagcaaagaaagggatcagcagaccaatcacaggaaagggcccagatgtgcacccactgtgcgtggacactgactcatgcacagaccgtgtacacagtggggaaggggcaggtgtggacactgactcgtgcacggaccgtgcacacagtggggaaggagcaggagctc

SEQ ID NO: 152 - DNA, 153 bp, Homo sapiens
gagctcatatgtatcctggaacttaaagtgaaaaaaattaaaaaataaagtaaaaaataataattttgagcaaaaaaaattagctgggcatggtggtgcatgtctgtggtcacagctgctcaggaggctgaagtgggaggatcacttgagctc

SEQ ID NO: 153 - DNA, 338 bp, Homo sapiens
gagctcatatgtatcctcactgggcctcagtttcctaatctgagaaatgagattcttggattaaataatgtagaaagttctttccagctggaaaaaatatatgagtcgaatatacttataggattagaatgcaggccgggcatggtggctcacgcctgtaatcccagcaccgtgggaggccaaggtgggagtatcacttcagctcaggggttcaagaccagcctgggcaacatagcaaaaccccatctctacaaaaaatacaaaaattagctgggcatggtggcgcaccctgtggtcacagctactcaggagcctgagcgggaggatcacttgagctc

SEQ ID NO: 154 - DNA, 148 bp, Homo sapiens
gagctcacacgatggacaccacttgccgacaccagcttggttggggataccctaacccagcagcactagaggaattaaacacacacacagaaatatagaggtgtgaagtgggaaatcggggtctcatagccttcagagctgagagctc

SEQ ID NO: 155 - DNA, 296 bp, Homo sapiens
gagctcgcataccacaggtgtggatgcaaaaaggctgttttaccgtctgccctcattggcggagagcaaccacctcatgcgaaaaagtgttaacttagctgttaacacttaagccacccgcacatggcaaagctaaaaagagcactgactgtaacactccttctgggactttgggggtcatgggtactcccctctagatgctgctgtgaggcccacatggacttttgcccctctgagcatcccaaagtactcaccccagctcctgtacctgctcacctcctgtgagggttgagctc

SEQ ID NO: 156 - DNA, 43 bp, Homo sapiens
gagctcgcataccacatggagctctctgtgtgagggtgagctc

SEQ ID NO: 157 - DNA, 44 bp, Homo sapiens
gagctcgtgtctcctcctgtgagctcgtgtctctggatgagctc

SEQ ID NO: 158 - DNA, 41 bp, Homo sapiens
gagctcaagtagtcctttttgtttgggtgtgctatgagctc

SEQ ID NO: 159 - DNA, 41 bp, Homo sapiens
gagctcaagtagtcctttgcagaaaggtgtgctatgagctc

SEQ ID NO: 160 - DNA, 784 bp, Homo sapiens
gagctcaccacagctcacacaccggctgtgtcagactgcagacagagtgcctcttcagcctaccttgacccatcctcctcactaggcagggcctcccctgcagggacttccaagaacctccagccaggggctcagggacagaactctgatctccctggactggagccctttagggagaagggatggccacagtctctatgaatcagcacacttagtctttactcctactaggtctgaggtatctgggcagtgcagactagtgtgttttcccccccagcaaagcacacccacctccaccaagggacaaagtgctttgttaaacaggtcctgctccctgtggcacccaactctgtctcaaaaacacacaaacaaacaaaaaaattgctgcatagtattccattgtatgagtagtaacacaacaatttttataatgcatagtattccattgtatgaatagtaatgtagcactatttgtttatacatttttatgattaaaaaacaaaatgtttttctattatgaataaagtggcaatgaatatttttgtacaagtgttttggtagctatacagttattgtcacttaatatatgcaattcgataggccagtcattcaaaatagaagatatacaaggtaggccgggcgtggtggctcacgcctgtaatctcagcactttgggaggccgaggtgggtggatcacctgtggttaggagtttcagaccagcctgaccaacatggagaaacctcatctctactaaaaatacaaaagtagctgagcgtggagctc

SEQ ID NO: 161 - DNA, 49 bp, Homo sapiens
gagctcttcaggtcagaagttgttccaagccggaacaccgtcagagctc

SEQ ID NO: 162 - DNA, 840 bp, Homo sapiens
  modified_base (561)..(593): a, c, g, t, unknown or other
gagctcagggaggacagaaacctcttgtggagcagaaggacaaaagctcgcttgattgtgattttcagtgcgaatacagactgaaagccgggtttgacgatcattctgacgtttttaagcaggagctgttaggaagttaccacaagggataactggctcgttgcggccaagcgttcatagggatgttgctttttgatcctttaatgttggttcttcctatcattgtgaagtagaattcaccaagcattggattgttcacccactaatggggcatgtgagctggatttaagaccttcgtgagacaggttagtttttaccctccgtgatgtgtgttgttgccatggtaatcctgctcagcatgagaggaatcacaggttcagacatgtgcttggttgaggagccaatggggtgaagctacagtctgttggattgtgactgaacgcctctaagtcagaatcccgcccaagaggaaggatacatcagcgccctggagccttggtgtcttgctagccgtccccgccccccgctggtgggctgctcgccctggggtgggggtgtgcnnnnnnnnnnnnnnnnnnnnnnnnnnnnnnnnnacctgcttcgggttggagttggccacaccaggccaacctccattgtggtgctcctgggtcaggcctcggggtcgcccagctgacgcctgagggtgtgagacagctggcggctcgccatgtccgcaggtcgaccagcaggccctctgaccgagcggtgtgcaggagtgcccgttgggatgagctggatccggagcgtccgcatctcggtcggaacctccagggtcaaccagctgccgcccgcgagctc

SEQ ID NO: 163 - DNA, 45 bp, Homo sapiens
gagctctgactatggatgtgagctactgaccatactggggagctc

SEQ ID NO: 164 - DNA, 922 bp, Homo sapiens
  modified_base (534)..(567): a, c, g, t, unknown or other
gagctctgactatggagtaacccttcttttattcttttacttttctagtaaacttgcttccactttactctatggacttgccctgaattctttcttgtgcaagatccaagaaccctctcttggggtctggatcgggaccgctttcctgtgatatcaccactctaactttcaaactatatttttaacagacaccaatgtttcatttttcctttatgtttcatgctttattttatattgaaaatttacatggggtgttttctttgctagaatattctccttttatatgttctcttttactacccatttatttttaaccatagtttatatatcacttctcaaggaagacttttctctcctcttggagagtgccactctccctaatgtatgttttcacagtttccttttttttccctatattctatctatctcatttcattgtaaaacaatttgttaacattcttgtgattcttttcttgggtcttagcctcatgaggaccaaaaaagctgccccctgccatgcttgccagaggcacnnnnnnnnnnnnnnnnnnnnnnnnnnnnnnnnnnctcagagacaaaagtgcttgcagcttccagagccagagagtgttatacaagaagtttcgatcttgtcgggtgttgagaggagcttctaaccccttggattccagggattgcatgactttgttatcctgctgactctggcccacacctgaccttatgctaaaagcaggactcagcatgaagctgtcacaggactatgactaatgacgtgagtgtaattgaggaatgagccacgggatattgcctaacttcaaaaaagagggaagctggagatgagtccaaccacatcacatgggcaaggattccatcagtcatgctaggtaatggaaccccaatacaaatccatactggggagctc

SEQ ID NO: 165 - DNA, 381 bp, Homo sapiens
gagctccgtatcacgctgcaagccttgtaagcttaaggggtggctcccatccctgggcagaatccacaccgtgattcttctgatgttttgggatacaaaagtagcaagagcctcccccaccccagagaatccttcaaggcatttctcccccatttttttatgacacccaaaacagctctgagatgcccactgccccaccaaagctggagttcttcgaaaggatgttctccatctcggaatctttctggaaatgttttccctccacatttctgacctcactatcaatgccttatgtgtgcaatcaagttaaactgaaatgtactcagtgaggcttctacttcccagccccttgctttgtgaacctgaggctgaggtgagctc

SEQ ID NO: 166 - DNA, 135 bp, Homo sapiens
gagctcctgaccttgtgatctgcccgcctcggcctcccaaagtgctgggattacaggtgtgagccacgtgcccagccaagataggtgtagtttttataatccctcacagctcaggagtcatgtgtgccagagctc

SEQ ID NO: 167 - DNA, 206 bp, Homo sapiens
gagctctgctagatgatgcaaatctacgcaagaatgaactggagacagagaataggtagagtattataggtggccgcatctggctataagttttccctatttgccttgattctgaatcccttaaacaaagaactggcagggcatggtggctcacgcctgtaatcccagcactttgggaggcccaggagggcagatcgcttgagctc

SEQ ID NO: 168 - DNA, 234 bp, Homo sapiens
gagctctactgttgctcagtaaagctcctcatcatcttactcactctccacttatccacatacttcattcttcctggacgcaggacgagaacttgggacctgccaaatggcagggctaaaagagctgtaacacaagtagggctgaaacatgccccttgctcgcctcaatgtgggtgacaagaaggagaggggagaagagctgcagccctttggggaacccagacctaggagctc

SEQ ID NO: 169 - DNA, 241 bp, Homo sapiens
gagctctactgttgctcaataaagcacctcttcaccttgctcaccttctacttgcccacatacctcattcttcctggactcaggacaagaactcgggacctgccaactagcagggctgaaagaggtgtaacataaacagggctgaaacgcaccccttgcttaccaaattgcaggcaagaagaagagaagagagaaggagacaagagctgtggcccttcagggagcccagacctaggagctc

SEQ ID NO: 170 - DNA, 345 bp, Homo sapiens
gagctccagcagccacagcctagccctggggaggctttgtggtctctgagggggcaggtgcactcccccaactccagttcatgtttttccctccaactctaagcctttttcttcctctgctattacccaggcaccctaccctgtcaacaacactggccttcaagaccctttgtagcataactcccacctataactcccacctgaagccagcccttcccacctctgcgcctctgatgcccaggacagctctgaccttgggcagccctgatctggggcagctctgacctcgagtagctgtgaccctgggcagctctgactctgggtagtcgtgaccctggggagctc

SEQ ID NO: 171 - DNA, 377 bp, Homo sapiens
gagctccagcagccacagcccagccctggggaggctttgtggtctctgagggggcaggtgcactcccccaactccagttcatgtttttccctccaactctaagcctttttcttcctctgctattacccaggcaccctaccctgtcaacaacactggccttcaagaccctttgtagcataactcccacctataactcccacctgaagccggcccttcccacctctgcgcctctgatgcccaggacagctctgaccgtgggcagctctgacccaggacagctctgaccttgggcagccctgatccggggcagctctgacctcgagtagctgtgaccctgggcagctctgactctgggtagtcgtgaccctggggagctc

SEQ ID NO: 172 - DNA, 409 bp, Homo sapiens
gagctccagcagccacagcccagccctggggaggctttgtggtctctgagggggcaggtgcactcccccaactccagttcatgtttttccctccaactgtaagcctttttcttcctctgctattacccaggcaccctaccctgtcaacaacactggccttcaagaccctttgtagcataactcccacctataactcccacctgaagccagcccttcccacctctgcgcctctgatgcccaggacagctctgaccgtgggcagctctgacccaggacagctctgaccttgggcagctctgacccgggacagctctgaccttgggcagccctgatctggggcagctctgacctcgagtagctgtgaccctgggcagctctgactctgggtagtcgtgaccctggggagctc

SEQ ID NO: 173 - DNA, 241 bp, Homo sapiens
gagctctactgttgctcaataaagcacctcttcaccttgctcaccttctacttgcccacatacctcattcttcctggactcaggacaagaactcgggacctgccaactagcagggctgaaagaggtgtaacataaacagggctggaacgcaccccttgcttaccacattgcaggcaagaagaagagaagagagaaggagacaagagctgtggcccttcagggagcccagacctaggagctc

SEQ ID NO: 174 - DNA, 220 bp, Homo sapiens
gagctctctccgtgcggaccgctgactccctctaccttgggttccctcggccccaccctggaacgccgggccttggcagattctggcccttcctggcccttcagtcgctgtcagaaaccccatctcatgctcggatgccccgagtgactgtggctcgcacctctccggaaacattggaaatctctcctctacgcgcggccacctgaaaccacaggagctc

SEQ ID NO: 175 - DNA, 90 bp, Homo sapiens
gagctcgagctcaggcccacaaggacagcagagaaatggaagggagtgggaaccgtgtcattttcttaccagaagacttagtcagtgctc

SEQ ID NO: 176 - DNA, 705 bp, Homo sapiens
  modified_base (395)..(436): a, c, g, t, unknown or other
gagctcgcgggcggcagctggtcgaccccggaggtgccgaccgagacggggacgcggcgggtccggctcgtcccgacgggcactcttacacgccgctcggtggagaaggcccgccggtcgacccgggacacggcgagacagcggctaagtctcaagagccgagaaggcaccaaggaaggggagagagggagggcggggagagagagagagagagagagagagagagcagagagagcagagagaagagagagagagagagggcgagagcgagagacagagagagagagagagagagagagagagagagagagagacgcgagcgagagagagagcagagagcgagagagagagaagagagagagagagagagagagagagagagagagagaaaaaannnnnnnnnnnnnnnnnnnnnnnnnnnnnnnnnnnnnnnnnntactctaagcaaaaacgacgagcattactggctgacggccagcagtggattccttttaaaagtcccgccgacgcaaacccgcggtggggctgaaaaaaattttaggagggggagttccgcgtggtcccagctcctaccgtcgggccgaggcccgtggaggtcgcgggcgcgtgaaccgggaatcggcaccctcgcgtcgtgcgccgcagcgtccggcggccgctgctgtcgacccggacacgtgcagacgccggctaagtccggagctc

SEQ ID NO: 177 - DNA, 405 bp, Homo sapiens
gagctcgcgggcggcagaccgcggcgctctatgaggcggaaagcgctcggcccggagccggcttgccgtgttcaccccggccctcctcgaggcggccacccgcgcgcttcaccatcgaacaggattgcgccgcgcggtgcagcgcggagaattcgagctggttttccagcccgaggtcaatctgcggacgtgcgaggtcgagctcgtcgaagcgctgttgcgctggaggttgccggacggacgcctgatcacaccggcggagtttcttcccgtcgccgaggcctccggcctgattctgcagatcaacgattgggtcgtgaagcgtgcgatcgaaacggccgcccggtggcacaacggttcgtggccggaggttcgtgttgcgatcaacgtgtcctccctgagctc

SEQ ID NO: 178 - DNA, 601 bp, Homo sapiens
gagctcgcgggcggcaccaagagtcgccgagcctacctccgcatcgcctgtacgccaccctgacccccaccctgtccctgcaggaaccttccccatccctgaagatgctggagagggaacttcacatctgtggggcctcctccagcatccctggctgaggatgcccacggcagagacatcccagcacatggatgccagctcagagagggctcctagacccagaacctaggcctcttttcatctacttctgtataaaagaagctggcagcttcctcccccatcctgagccactatatgcttaagatcccaggagtcagggagggaaccctgttcccccaagtctgcctagtgaggccccttctttggagaagaggaaaatttcccgggcaaaggccaggtgggcccatcaacagtaccctgggtctgcactgccacccagtggctacagtgtagcacatcaggttctgatcctgcccgcacctgactgccttgggaccctctcgcttctcccagacctgcgcaagaacttcgatcaggagcctctgggcaaggaggtgcccctggaccatgaaatacgactgccagatagggcgaagaggtc

SEQ ID NO: 179 - DNA, 19 bp, Homo sapiens
gcctccctcgcgccatcag

SEQ ID NO: 180 - DNA, 19 bp, Homo sapiens
gccttgccagcccgctcag

SEQ ID NO: 181 - DNA, 17 bp, Homo sapiens
gagctcccccttcatga

SEQ ID NO: 182 - DNA, 16 bp, Homo sapiens
gccctaacgagagctc

SEQ ID NO: 183 - DNA, 16 bp, Homo sapiens
gagctccctcaatttc

SEQ ID NO: 184 - DNA, 16 bp, Homo sapiens
ttaggcttgtgagctc

SEQ ID NO: 185 - DNA, 16 bp, Homo sapiens
gagctcacagggctga

SEQ ID NO: 186 - DNA, 16 bp, Homo sapiens
tacattctaggagctc

SEQ ID NO: 187 - DNA, 16 bp, Homo sapiens
gagctctcatgtctgg

SEQ ID NO: 188 - DNA, 16 bp, Homo sapiens
tctgcctgccgagctc

SEQ ID NO: 189 - DNA, 16 bp, Homo sapiens
gagctctcatgccttt

SEQ ID NO: 190 - DNA, 16 bp, Homo sapiens
tttgctcccggagctc

SEQ ID NO: 191 - DNA, 16 bp, Homo sapiens
tccctggtttgagctc

SEQ ID NO: 192 - DNA, 134 bp, Homo sapiens
gatctcctgaccttgtgatctgcccgcctcggcctcccaaagtgctgggattacaggtgtgagccacgtgcccagccaagataggtgtagtttttataatccctcacagctcaggagtcatgtggccagagctc

SEQ ID NO: 193 - DNA, 55 bp, Homo sapiens
gagctcctgacctcaggtgatccacccatctcggcctcccaaagtgctgggatta

SEQ ID NO: 194 - DNA, 115 bp, Homo sapiens
ggcagtccagttccggcgtcactcggtactaacgtggtgacgtgaggtcggagccactatctcactctgggacagagtttttgttttgttccggtccacgtcaccgagtacggat

SEQ ID NO: 195 - DNA, 115 bp, Homo sapiens
ggcagtccagttccgacgtcactcggtactaacgtggtgacgtgaggtcggagccactatctcactctgggacagagtttttgttttgttccggtccacgtcaccgagtacggat

SEQ ID NO: 196 - DNA, 30 bp, Homo sapiens
ccccccacctagtggactccagtcctcgag

SEQ ID NO: 197 - DNA, 138 bp, Homo sapiens
ccctcagcctcccaaagtgttgggattacaggtgtgagccacagtacccggcctgactgactaaataaatgaatgaaatattcgtggaatgaatgagccaggattcctccctgcttttctctcctgacctgacggagc

SEQ ID NO: 198 - DNA, 138 bp, Homo sapiens
ccctcagcctcccaaagtgttgggattacaggtgtgagccacagtacccggcctgactgactaaataaatgaatgaaatattcgtggaatgaatgagccaggattcctccctgcttttctctcctgatctgatggagc

SEQ ID NO: 199 - DNA, 45 bp, Homo sapiens
gagctcaggagttcgagaccattctgggcaacatggcaagacccc

SEQ ID NO: 200 - DNA, 45 bp, Homo sapiens
gagctcaggagttcgagaccagcctgggcaacatggcaagacccc

SEQ ID NO: 201 - DNA, 101 bp, Homo sapiens
cccaccatatctacaaatatatatataagaaaattagccgggcgtgctggctcactcctgcaatcccagcactttgggaggccgaggtggtcgaagagctc

SEQ ID NO: 202 - DNA, 24 bp, Homo sapiens
tcctttagttccgtggcgctcgag

SEQ ID NO: 203 - DNA, 20 bp, Homo sapiens
ctcgagacctgaccacgtac

SEQ ID NO: 204 - DNA, 19 bp, Homo sapiens
ctcgagacctgacacgtac